(19) United States
(12) Patent Application Publication
(10) Pub. No.: US 2024/0301459 A1
(43) Pub. Date: Sep. 12, 2024

(54) METHODS FOR PROCESSING NUCLEIC ACID SAMPLES

(71) Applicant: 10x Genomics, Inc., Pleasanton, CA (US)

(72) Inventor: Xinying ZHENG, San Jose, CA (US)

(21) Appl. No.: 18/655,106

(22) Filed: May 3, 2024

Related U.S. Application Data
(63) Continuation-in-part of application No. 18/410,457, filed on Jan. 11, 2024.
Application No. 18/410,457 is a continuation of application No. 17/522,741, filed on Nov. 9, 2021, now abandoned.
(60) Provisional application No. 63/111,821, filed on Nov. 10, 2020.

(51) Int. Cl. C12P 19/34; C12N 15/10; C12Q 1/6806

(52) U.S. Cl. CPC C12P 19/34; C12N 15/1065; C12Q 1/6806

(57) ABSTRACT

Provided are systems and methods for analyzing a single cell application or experiment. A set of control beads may be introduced to a biological sample and subjected to the single cell application. The control beads may be configured to mimic analytes in the biological sample, such as a cell or other analyte, and comprise one or more known sequences. The one or more known sequences may be identified to analyze the single cell application.

### CROSS-REFERENCE

This application is a continuation-in-part of U.S. patent application Ser. No. 18/410,457, filed on Jan. 11, 2024, which is a continuation of U.S. patent application Ser. No. 17/522,741, filed on Nov. 9, 2021, which claims the benefit of U.S. Provisional Application No. 63/111,821, filed Nov. 10, 2020, each of which is entirely incorporated herein by reference for all purposes.

### SEQUENCE LISTING

The instant application contains a Sequence Listing which has been submitted electronically in XML format and is hereby incorporated by reference in its entirety. Said XML copy, created on Apr. 15, 2024, is named 43487-868501 SL_15042024 and is 9,560 bytes in size.

### BACKGROUND

A sample may be processed for various purposes, such as identification of a type of moiety within the sample. The sample may be a biological sample. Biological samples may be processed, such as for detection of a disease (e.g., cancer) or identification of a particular species. There are various approaches for processing samples, such as polymerase chain reaction (PCR) and sequencing. The processing may be single cell processing, such as to yield data specific and distinct to individual cells.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Biological samples in partitions may be subjected to various processes, such as chemical processes or physical processes. Samples in partitions may be subjected to heating or cooling, or chemical reactions, such as to yield species that may be qualitatively or quantitatively processed.

### SUMMARY

Single cell processing experiments are becoming increasingly common and efficient. Massively parallel sequencing or next-generation sequencing, for example, allow for high-throughput processing that can yield thousands, tens of thousands, hundreds of thousands, or millions of, or more base reads in a single run. However, as is often the case, the results (e.g., data) from an experiment are only as reliable as the experimental process itself can be validated, that is, that the experiment performed as designed. For example, experiments may have different sensitivities, accuracies, and/or biases, which are not necessarily, if at all, reflected in the data results. Given the relatively miniscule scale and high throughput of a single cell assay, as well as the irreproducible nature of some of these assays which can consume or contaminate unique samples during an immediate assay, it is difficult to validate or investigate, or otherwise perform quality control (QC) on, how well an experiment performs during the single cell assay or be able to compare two or more different single cell assays, or results thereof.

One approach for quality control of a single cell sequencing experiment may involve introducing a control into the sample to be sequenced, and tracking the control. For example, the External RNA Controls Consortium (ERCC) has developed ribonucleic acid (RNA) control transcripts (ERCC RNA Spike-In Controls) with known sequences that may be spiked into RNA samples for input into a RNA sequencing (RNA-seq) experiment. The results of a spiked-in RNA-seq experiment may then be compared to the known control sequences, such as to determine whether the experiment was able to properly detect the control, or at what dosages of controls the experiment was able to detect the control to assess sensitivity, and the like. Another approach for quality control may involve introducing an exogenous genome or epigenome as reference into the sample, such as for normalizing chromatin immunoprecipitation sequencing (ChIP-seq) analyses. However, such methods of control fail to mimic true single cell behavior—it would be more accurate to describe the above methods as performing quality control on the treatment of an analyte in a cell (e.g., nucleic acids, chromatins, etc.), and not the cell, in the single cell experiment. For example, the known nucleic acids (e.g., known transcripts) are distributed uniformly in equal amounts to all partitions, including partitions that already contain cells, inherently biasing the single cell experiment. Furthermore, using bulk ERCC controls require carefully controlling the input concentration and subsampling to manage sequencing requirements, both of which introduce handling variables that can affect quality control. Additionally, using bulk ERCC controls does not allow for quality control of the microfluidics involved in the single cell experiment.

Recognized herein is a need for systems and methods for quality control of single cell processing experiments that addresses at least the abovementioned problems. Provided are synthetic cells that mimic single cell behavior in single cell processing experiments. A synthetic cell may be a bead comprising one or more known sequences. The bead may be a gel bead. The synthetic cell may be introduced into a cell sample and have approximately the same or substantially the same size as other cells in the cell sample. The synthetic cell may be carried through the entire workflow of a single cell processing experiment with the cell sample. After sequencing, the one or more known sequences in the synthetic cell may be identified and analyzed to determine various characteristics of the single cell processing experiment, such as the effectiveness or the efficiency of the library preparation process or the sequencing process.

In an aspect, provided is a method for analyzing a single cell process in a cell sample, comprising: (a) providing a plurality of analyte carriers, a plurality of control beads, and a plurality of barcode beads; (b) generating a plurality of partitions comprising a first partition and a second partition, wherein (i) the first partition comprises an analyte carrier from the plurality of analyte carriers and a first barcode bead from the plurality of barcode beads, wherein the analyte carrier comprises a template nucleic acid molecule, and wherein the first barcode bead comprises a first nucleic acid barcode molecule comprising a first barcode sequence, and (ii) the second partition comprises a control bead from the plurality of control beads and a second barcode bead from the plurality of barcode beads, wherein the second barcode bead comprises a second nucleic acid barcode molecule comprising a second barcode sequence, wherein the second barcode sequence is different than the first barcode sequence, and wherein the control bead comprises a nucleic acid molecule comprising a known sequence; (c) using (i) the template nucleic acid molecule and the first nucleic acid barcode molecule to generate a barcoded template nucleic acid molecule and (ii) the nucleic acid molecule comprising the known sequence and the second nucleic acid barcode molecule to generate a barcoded nucleic acid molecule comprising the known sequence; and (d) sequencing the barcoded template nucleic acid molecule, or derivative thereof, and the barcoded nucleic acid molecule, or derivative thereof wherein (i) a sequence of the barcoded template nucleic acid molecule identifies the analyte carrier, and (ii) a sequence of the barcoded nucleic acid molecule identifies the control bead.

In some embodiments, the method further comprises identifying the known sequence to analyze the single cell process.

In some embodiments, the method further comprises providing a plurality of second control beads, wherein the plurality of partitions comprises a third partition, wherein the third partition comprises a third barcode bead from the plurality of barcode beads and a second control bead from the plurality of second control beads, wherein the third barcode bead comprises a third nucleic acid barcode molecule comprising a third barcode sequence, wherein the third barcode sequence is different than the first barcode sequence and the second barcode sequence, and wherein the second control bead comprises a second nucleic acid molecule comprising a second known sequence, wherein the second known sequence is different from the known sequence.

In some embodiments, the method further comprises generating a second barcoded nucleic acid molecule comprising the second known sequence, wherein a sequence of the second barcoded nucleic acid molecule identifies the second known sequence. In some embodiments, the method further comprises processing the known sequence and the second known sequence to analyze the single cell process. In some embodiments, the method further comprises processing comprises comparing a frequency of the known sequence and a frequency of the second known sequence to analyze the single cell process. In some embodiments, the processing further comprises determining a doublet rate.

In some embodiments, the second control bead has a size within about 70% deviation from an average size of the analyte carrier. In some embodiments, the second control bead has a size within about 40% deviation from an average size of the analyte carrier. In some embodiments, the second control bead has a size within about 25% deviation from the average size of the analyte carrier. In some embodiments, the second control bead has a size within about 10% deviation from the average size of the analyte carrier. In some embodiments, the second control bead has a size within about 5% deviation from the average size of the analyte carrier.

In some embodiments, the size of the second control bead has a size within about 40% deviation from an average size of the control bead. In some embodiments, the second control bead has a size within about 25% deviation from the average size of the control bead. In some embodiments, the second control bead has a size within about 10%deviation from the average size of the control bead. In some embodiments, the second control bead has a size within about 5% deviation from the average size of the control bead.

In some embodiments, the known sequence is derived from a first species and the second known nucleic acid sequence is derived from a second species. In some embodiments, the first species is a human and wherein the second species is a mouse.

In some embodiments, the control bead has a size within about 40% deviation from an average size of the analyte carrier. In some embodiments, the control bead has a size within about 25% deviation from the average size of the analyte carrier. In some embodiments, the control bead has a size within about 10% deviation from the average size of the analyte carrier. In some embodiments, the control bead has a size within about 5% deviation from the average size of the analyte carrier.

In some embodiments, the control bead has a size between from about 15 micrometers to about 35 micrometers. In some embodiments, the control bead has a size between from about 35 micrometers to about 60 micrometers.

In some embodiments, a given bead from the plurality of barcode beads comprises a plurality of nucleic acid barcode molecules comprising a common barcode sequence. In some embodiments, the common barcode sequence is different from common barcode sequences of other beads of the plurality of beads.

In some embodiments, the method further comprises, prior to (b), mixing analyte carriers from the plurality of analyte carriers with control beads of the plurality of control beads.

In some embodiments, the method further comprises, prior to (b), mixing analyte carriers from the plurality of analyte carriers with control beads of the plurality of control beads and control beads of the second plurality of control beads. In some embodiments, the control beads from the plurality of control beads is present in the mixture at a first concentration, wherein the control beads from the second plurality of control beads is present in the mixture at a second concentration, and wherein the first concentration and the second concentration are known. In some embodiments, a ratio of the first concentration to the second concentration is at least about 1:0.001. In some embodiments, a ratio of a concentration of the analyte carriers in the mixture to the first concentration is known. In some embodiments, the ratio is about 0.001:1.

In some embodiments, the control bead comprises a plurality of unique nucleic acid molecules with known nucleic acid sequences that vary in length, concentration, and/or GC content. In some embodiments, the plurality of unique nucleic acid molecules is a plurality of DNA molecules. In some embodiments, the plurality of unique nucleic acid molecules is a plurality of RNA molecules.

In some embodiments, (i) the control bead comprises a first plurality of unique nucleic acid molecules with known nucleic acid sequences that vary in length, concentration, and/or GC content, and wherein (ii) the second control bead comprises a second plurality of unique nucleic acid molecules with known nucleic acid sequences that vary in length, concentration, and/or GC content, wherein the first plurality of unique nucleic acid molecules and the second plurality of unique nucleic acid molecules are different.

In some embodiments, the first plurality of unique nucleic acid molecules and the second plurality of unique nucleic acid molecules are both a plurality of DNA molecules. In some embodiments, the first plurality of unique nucleic acid molecules and the second plurality of unique nucleic acid molecules are both a plurality of RNA molecules.

In some embodiments, a given bead from the plurality of barcode beads comprises a plurality of nucleic acid barcode molecules, wherein each of the plurality of nucleic acid barcode molecules comprises an identifier sequence, wherein the identifier sequence is different from identifier sequences associated with other nucleic acid barcode molecules in the plurality of nucleic acid barcode molecules. In some embodiments, the method further comprises identifying a first set of identifiers associated with the first plurality of unique nucleic acid molecules and a second set of identifiers associated with the second plurality of unique nucleic acid molecules. In some embodiments, the method further comprises processing the first set of identifiers and the second set of identifiers to determine an identifier purity for the single cell process.

In some embodiments, a given bead from the plurality of barcode beads comprises a plurality of nucleic acid barcode molecules releasably attached thereto. In some embodiments, the first nucleic acid barcode molecule and the second nucleic acid barcode molecule is released from the first barcode bead and the second barcode bead.

In some embodiments, the plurality of barcode beads is a plurality of gel beads.

In some embodiments, the nucleic acid molecule is releasably attached to the control bead. In some embodiments, the nucleic acid molecule is released from the control bead.

In some embodiments, the nucleic acid molecule is within the control bead.

In some embodiments, the control bead comprises a first functional group, and said nucleic acid molecule comprising the known sequence comprises a second functional group. In some embodiments, the nucleic acid molecule comprising the known sequence is attached to said control bead by reacting said first functional group with said second functional group. In some embodiments, the first functional group is an alkyne, a trans-cyclooctene, or an avidin, or any combination thereof. In some embodiments, the first functional group is an alkyne. In some embodiments, the second functional group is an azide, a tetrazine, or a biotin, or a combination thereof. In some embodiments, the second functional group is an azide. In some embodiments, the first functional group reacts with said second functional group in a click reaction. In some embodiments, the click reaction is a copper-catalyzed azide-alkyne cycloaddition reaction, an inverse-electron demand Diels-Alder reaction, or an avidin-biotin interaction. In some embodiments, the click reaction is a copper-catalyzed azide-alkyne cycloaddition reaction.

In some embodiments, said control bead is attached to said nucleic acid molecule comprising the known sequence using bioconjugation chemistries other than click chemistry. Thus, in some embodiments, said first functional group is a carboxylic acid, and said second functional group is an amine. Said first functional group can react with said second functional group via an amide bond formation reaction. Such amide bond formation reaction can further comprise 1-ethyl-3-(3-dimethylaminopropyl)carbodiimide (EDC), N-hydroxysuccinimide (NHS), or a combination thereof.

In some embodiments, the known sequence is synthetic.

In some embodiments, the known sequence is derived from a biological sample.

In some embodiments, the control bead comprises a protein-DNA complex and the nucleic acid molecule is part of the protein-DNA complex. In some embodiments, the known sequence comprises defined protein binding sites. In some embodiments, the single cell process comprises ATAC-seq.

In some embodiments, the plurality of analyte carriers is a plurality of cells. In some embodiments, the plurality of analyte carriers is a plurality of nuclei. In some embodiments, the plurality of analyte carriers is a plurality of cell beads.

In some embodiments, the plurality of partitions is a plurality of droplets. In some embodiments, the plurality of partitions is a plurality of wells.

In another aspect, provided is a kit for analyzing a cellular sample comprising analyte carriers, comprising: a set of control beads, wherein at least a subset of said set of control beads comprise (i) identical nucleic acid molecules each comprising a known nucleic acid sequence, and (ii) has a characteristic corresponding to within 70% deviation from an average value of said characteristic of said analyte carriers; and an index comprising said known nucleic acid sequence.

In some embodiments, said characteristic is selected from a group consisting of size, shape, density, conductivity, hardness, deformability, and hydrophobicity.

In some embodiments, the index comprises a concentration of the known nucleic acid sequence.

In some embodiments, the set of control beads comprises a first subset of control beads and a second subset of control beads. In some embodiments, the first subset of control beads comprises a first set of nucleic acid molecules comprising a first set of known sequences, and said second subset of control beads comprises a second set of nucleic acid molecules comprising a second set of known sequences. In some embodiments, the first set of known sequences comprises 96 different known sequences and said second set of known sequences comprises 96 different known sequences. In some embodiments, the 96 different known sequences of said first set of known sequences are different then said 96 different known sequences of said second set of known sequences. In some embodiments, the first subset of control beads and said second subset of control beads are present in a ratio. In some embodiments, the ratio is at least about 1:0.001, 1:0.01, 1:0.1, 1:0.2, 1:0.3, 1:0.4, 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, or 1:1000. In some embodiments, the ratio is at most about 1:1000, 1;100, 1:90, 1:80, 1:70, 1:60, 1:50, 1:40, 1:30, 1:20, 1:10, 1:9, 1:8, 1:7, 1:6, 1:5, 1:4, 1:3, 1:2, 1:1.9, 1:1.8, 1:1.7, 1: 1.6, 1:1.5, 1:1.4, 1:1.3, 1:1.2, 1:1.1, 1:1, 1:0.9, 1:0.8, 1:0.7, 1:0.6, 1:0.5, 1:0.4, 1:0.3, 1:0.2, 1:0.1, 1:0.01, or 1:0.001. In some embodiments, the ratio is about 1:2.

In some embodiments, a subset of the control beads comprises additional identical nucleic acid molecules that are different than the identical nucleic acid molecules, each of which additional nucleic acid molecules comprises an additional known nucleic acid sequence. In some embodiments, the index comprises the additional known nucleic acid sequence. In some embodiments, the index comprises a concentration of the additional known nucleic acid sequence. In some embodiments, the known nucleic acid sequence and the additional known nucleic acid sequence vary in length, concentration, and/or GC content.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual analyte carriers.

FIG. 2 shows an example of a microfluidic channel structure for delivering barcode carrying beads to droplets.

FIG. 3 shows an example of a microfluidic channel structure for co-partitioning analyte carriers and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7A shows a cross-section view of another example of a microfluidic channel structure with a geometric feature for controlled partitioning. FIG. 7B shows a perspective view of the channel structure of FIG. 7A.

FIG. 8 illustrates an example of a barcode carrying bead.

FIG. 9 illustrates a method for performing quality control for a single cell experiment.

FIG. 10 illustrates two synthetic cells that may be used in specific, known ratios as sequencing standards

FIG. 11 illustrates a HiScribe RNA synthesis with capping and tailing starting from an RNA transcript.

FIG. 12A shows digital droplet PCR results showing ligated (azide-containing) and un-ligated RNA sequences derived from a first transcript. FIG. 12B illustrates digital droplet PCR results showing ligated (azide-containing) and un-ligated RNA sequences derived from a second transcript.

FIG. 13 illustrates the use of click chemistry to attach RNA sequences (e.g., azide-functionalized) produced from RNA transcripts onto gel beads (e.g., alkyne-functionalized) to generate a synthetic cell (e.g., a control bead). The click reaction may be a Cu-mediated azide-alkyne cycloaddition reaction.

FIG. 14A shows a fluorescence spectrum for a control click reaction that only contains an alkyne but no azide and thus where no click reaction products were expected. FIG. 14B shows a right shift of the fluorescence signal indicating an increase in fluorescence intensity confirming that alkyne-functionalized gel beads were functionalized with azide-modified RNA molecules via Cu-mediated azide-alkyne click reaction in a first test reaction. FIG. 14C shows a right shift of the fluorescence signal indicating an increase in fluorescence intensity confirming that alkyne-functionalized gel beads were functionalized with azide-modified RNA molecules via Cu-mediated azide-alkyne click reaction in a second test reaction. FIG. 14D shows a right shift of the fluorescence signal indicating an increase in fluorescence intensity confirming that alkyne-functionalized gel beads were functionalized with azide-modified RNA molecules via Cu-mediated azide-alkyne click reaction in a third test reaction.

FIG. 15 shows a diagram indicating signals from complementary DNA (cDNA) fragments that match the expected transcript sizes for both transcripts tested. The additional peak at about 75 bp may be due to a formed primer dimer.

FIG. 16 shows a bioanalyzer trace indicating that the expected transcripts were detected at approximately 311 bp and 674 bp. The observed peak width may be due to random fragmentation of the nucleic acid molecules.

FIG. 17 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated. Ranges may be expressed herein as from “about” one particular value, and/or to “about” another particular value. When such a range is expressed, another possibility includes from the one particular value and/or to the other particular value. Similarly, when values are expressed as approximations, by use of the antecedent “about,” it will be understood that the particular value may be another possibility. It will be further understood that the endpoints of each of the ranges are in relation to the other endpoint, and independently of the other endpoint. The term “about” as used herein may refer to a range that is 15% plus or minus from a stated numerical value within the context of the particular usage. For example, about 10 may include a range from 8.5 to 11.5.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The terms “bead,” “gel bead,” and “synthetic cell,” may be used interchangeably herein and generally refer to a particle that can mimic a cell. Such particle can be a bead such as a gel bead. The bead may be similar to a cell, e.g., a cell of a biological sample in terms of size, shape, etc. A bead may be a control bead or a barcode bead. A control bead may be structurally similar to a barcode bead. For example, in a control bead, at least some of the barcode sequence(s) of a barcode bead may be replaced with control sequences (e.g., nucleic acid sequences such as genes). This modular design of control and/or barcode beads may be advantageous and simplify production and/or quality control of such beads.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “analyte carrier,” as used herein, generally refers to a discrete biological system derived from a biological sample. The analyte carrier may be a macromolecule. The analyte carrier may be a small molecule. The analyte carrier may be a biological particle. The analyte carrier may be a virus. The analyte carrier may be a cell or derivative of a cell. The analyte carrier may be an organelle. The analyte carrier may be a rare cell from a population of cells. The analyte carrier may be any type of cell, including without limitation prokaryotic cells, eukaryotic cells, bacterial, fungal, plant, mammalian (e.g., from a human and/or a mouse), or other animal cell type, mycoplasmas, normal tissue cells, tumor cells, or any other cell type, whether derived from single cell or multicellular organisms. The analyte carrier may be a constituent of a cell. The analyte carrier may be or may include DNA, RNA, organelles, proteins, or any combination thereof. The analyte carrier may be or may include a matrix (e.g., a gel or polymer matrix) comprising a cell or one or more constituents from a cell (e.g., cell bead), such as DNA, RNA, organelles, proteins, or any combination thereof, from the cell. The analyte carrier may be obtained from a tissue of a subject. The analyte carrier may be a hardened cell. Such hardened cell may or may not include a cell wall or cell membrane. The analyte carrier may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from an analyte carrier. The macromolecular constituent may comprise a nucleic acid. In some cases, the analyte carrier may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

Provided herein are systems and methods for analyzing a single cell process in a cell sample. Such systems and methods may provide synthetic cells that mimic single cell behavior in single cell processing experiments. A synthetic cell may be a bead comprising one or more known sequences. For example, the bead may comprise a nucleic acid molecule (e.g., DNA, RNA, etc.) comprising a known sequence. The bead may be coupled to, or otherwise incorporate, the nucleic acid molecule. In some instances, the nucleic acid molecule may be captured by the bead (e.g., in a matrix). The bead may be a gel bead. The synthetic cell may be introduced into a cell sample and have approximately the same or substantially the same size as other cells in the cell sample (e.g., from about 15 micrometers (μm) to about 60 μm). The synthetic cell may be carried through the entire workflow of a single cell processing experiment with the cell sample. After sequencing, the one or more known sequences in the synthetic cell may be identified and analyzed to determine various characteristics of the single cell processing experiment, such as the effectiveness or the efficiency of the library preparation process or the sequencing process.

### Quality Control for Single Cell Processing

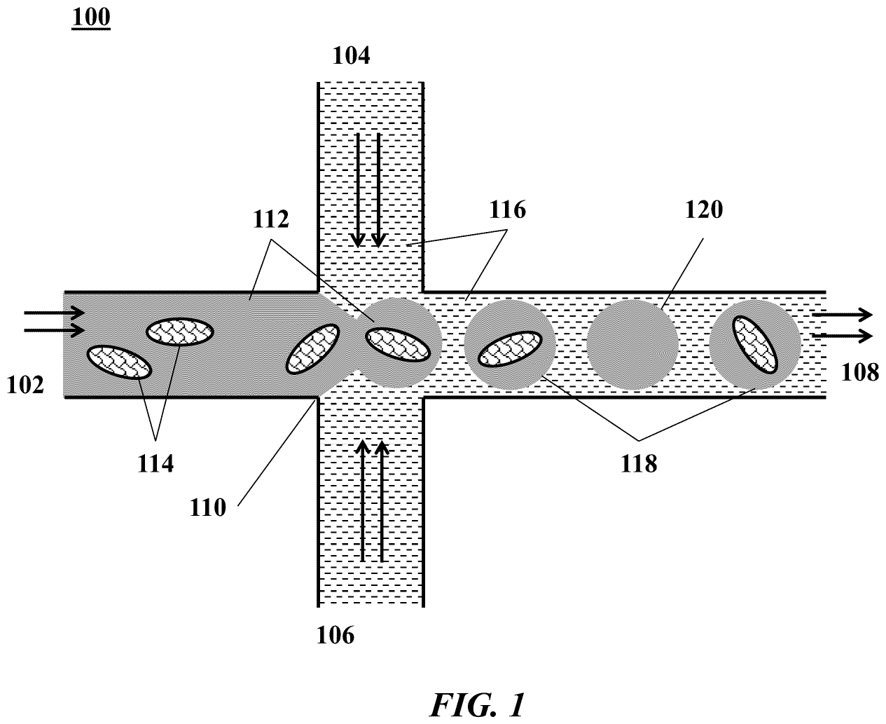

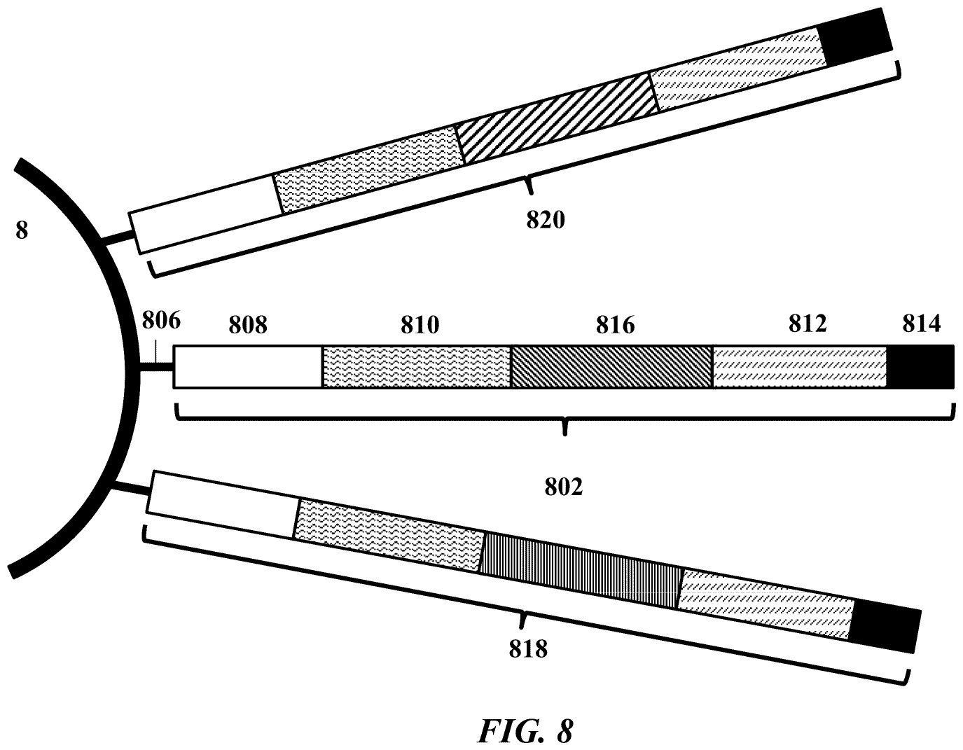

In an aspect, the present disclosure provides a method for analyzing a single cell process in a cell sample by distributing a set of barcode beads, a set of control beads, and the cell sample into a plurality of partitions. Systems and methods for generating partitions are described elsewhere herein, such as with respect to FIGS. 1-7B. The partitions may be droplets in emulsions or other microcapsules. The partitions may be solid partitions, such as wells. Barcode beads are described elsewhere herein, such as with respect to FIG. 8. The systems and methods may use more than one set of control beads. For example, there may be at least about 2, 3, 4, 5, 6, 7, 8, 9, 10, 15, 20, or more sets of control beads. Alternatively, there may be at most about 20, 15, 10, 9, 8, 7, 6, 5, 4, 3, 2, or 1 set of control beads.

The method may comprise generating the plurality of partitions including a first partition and a second partition. The first partition may comprise an analyte carrier, such as a cell, from the cell sample and a first barcode bead from the set of barcode beads. The analyte carrier may be or comprise a biological particle. The analyte carrier may be or comprise an analyte. The analyte carrier may comprise a template nucleic acid molecule. The first barcode bead may comprise a first barcode nucleic acid molecule comprising a first barcode sequence. The second partition may comprise a second barcode bead from the set of barcode beads and a first control bead from the set of control beads. The second barcode bead may comprise a second barcode nucleic acid molecule comprising a second barcode sequence. The second barcode sequence may be different from the first barcode sequence. The first control bead may comprise a nucleic acid molecule comprising a known sequence.

The method may comprise processing the first partition and the second partition to generate a barcoded template nucleic acid molecule and a barcoded nucleic acid molecule. The barcoded template nucleic acid molecule may comprise the first barcode sequence to identify the analyte carrier (e.g., cell). The barcoded nucleic acid molecule may comprise the second barcode sequence to identify the known sequence. The barcoded template nucleic acid molecule and the barcoded nucleic acid molecule may be sequenced to identify the analyte carrier and/or the known sequence to analyze the single cell process.

As described elsewhere herein, in some cases, the method may further comprise using a second set of control beads. For example, a third partition may be generated. The third partition comprises a third barcode bead from the set of barcode beads and a second control bead from the second set of control beads. The third barcode bead may comprise a third barcode nucleic acid molecule comprising a third barcode sequence. The second control bead may comprise a second nucleic acid molecule comprising a second known sequence. The second known sequence may be different from the known sequence. The third partition may be processed to generate a second barcoded nucleic acid molecule. The second barcoded nucleic acid molecule may comprise the third barcode sequence to identify the second known sequence. The second barcoded nucleic acid molecule may be sequenced, in addition to the barcoded template nucleic acid molecule and the barcoded nucleic acid molecule to analyze the single cell process.

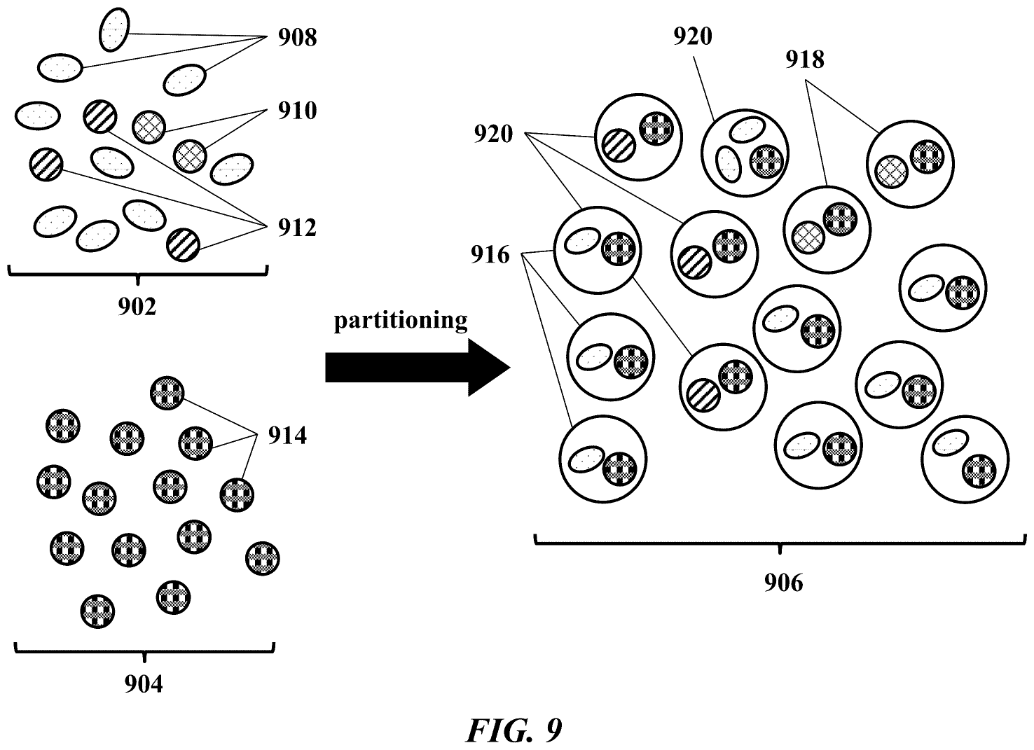

FIG. 9 illustrates a method for performing quality control for a single cell experiment. A mixed biological sample 902 and a set of barcode beads 904 may be partitioned into a plurality of partitions 906. The mixed biological sample 902 may comprise a plurality of cells 908, a plurality of first control beads 910, and a plurality of second control beads 912. The set of barcode beads 904 may comprise a plurality of barcode beads 914. Each partition in the plurality of partitions 906 may comprise a barcode bead and one cell of the plurality of cells of the biological sample, a first control bead, or a second control bead. For example, some partitions 916 may comprise a barcode bead and a cell, some partitions 918 may comprise a barcode bead and a first control bead, and some partitions 920 may comprise a barcode bead and a second control bead. It will be appreciated, as described elsewhere herein, that during partition generation, a partition may have no particles (e.g., beads, cells, etc.), have more than one barcode beads or more than one of a cell, a first control bead, or a second control bead, or any combination thereof. Systems and methods may be configured to generate mostly singularly-occupied partitions (e.g., greater than 50%, 60%, 70%, 80%, 90%, 95%, 96%, 97%, 98%, 99%, or higher, etc.) and/or configured to sort out singularly-occupied partitions for further processing. Singularly-occupied partitions may refer to those partitions having one of a barcode bead and/or one of a cell or control bead (e.g., first control bead, second control bead).

A barcode bead (e.g., 914) in the set of barcode beads 914 may comprise a barcode. An example of a barcode carrying bead is shown in FIG. 8. The bead may be coupled to a nucleic acid molecule, such as an oligonucleotide, by a releasable linkage (e.g., disulfide linker). The bead may be coupled to one or more other nucleic acid molecules. Each nucleic acid molecule may be or comprise a bead-specific barcode sequence (e.g., unique to each bead) that is common to all nucleic acid molecules coupled to the same bead. The barcode may comprise a number of sequence elements, such as the bead-specific barcode sequence, a functional sequence used for subsequent processing (e.g., flow cell attachment sequence, sequencing primer sequence, etc.), a priming sequence (e.g., poly-T sequence, targeted priming sequence, random priming sequence, etc.), an anchoring sequence (e.g., random short sequences, etc.), and/or a molecule-specific barcode sequence. The molecule-specific barcode sequence, also referred to as a unique molecular identifier (UMI), may be unique to each nucleic acid molecule coupled to the bead, even within the same bead. For example, for a barcode bead coupled to tens to hundreds of thousands or even millions of individual nucleic acid molecules, each individual nucleic acid molecule may be uniquely identified by the UMI. As will be appreciated, two or more UMIs may overlap. For example, the UMI count purity may measure chimerism in the PCR which contributes to significant technical noise at higher sequencing depths. Beneficially, the systems and methods described herein may determine a UMI purity rate for the single cell experiment.

A first control bead from the plurality of first control beads 910 may be a synthetic cell configured to mimic single cell behavior for a single cell processing experiment, such as for sequencing. The first control bead may be a bead, such as a gel bead. The first control bead may have approximately the same or substantially the same size as the average size of the plurality of cells 908 in the mixed biological sample 902. For example, the first control bead may have a diameter from about 15 micrometers (μm) to about 25 μm. Alternatively or in addition to, the first control bead may have a diameter from at least about 10, 11, 12, 13, 14, 15, 16, 17, 18, 19, 20, 21, 22, 23, 24, 25, 26, 27, 28, 29, 30, 35, 40, 45, 50, 55, 60, 65 μm or greater. Alternatively or in addition to, the first control bead may have a diameter of at most about 65, 60, 55, 50, 45, 40, 35, 30, 29, 28, 27, 26, 25, 24, 23, 22, 21, 20, 19, 18, 17, 16, 15, 14, 13, 12, 11, 10 μm or less. In some instances, the size (e.g., diameter) of a first control bead may deviate from an average size (e.g., diameter) of a cell in the mixed cell sample within at least about 90%, 80%, 70%, 60%, 50%, 40%, 30%, 25%, 20%, 15%, 10%, 9%, 8%, 7%, 6%, 5%, 4%, 3%, 2%, 1%, or lower. Alternatively or in addition to, the first control bead may have other physical characteristics or properties that mimic a single cell, such as density, mass, shape, and the like. Alternatively or in addition to, the first control bead may have other characteristics or properties, such as chemical properties, that mimic a single cell, such as conductivity, hardness, deformability, interactive properties with aqueous or non-aqueous solutions, and the like. In some instances, the first control bead may have optical properties that mimic a single cell, such as those that are discernable with flow cytometry (e.g., forward or side scatter).

The first control bead may comprise a first set of one or more known sequences. For example, the bead may comprise a nucleic acid molecule (e.g., DNA, RNA, etc.) comprising a known sequence. The first control bead may comprise any other analyte (e.g., proteins, metabolite, nucleic acid molecule, etc.) comprising a known sequence. In some instances, the bead may be coupled to, or otherwise incorporate, the nucleic acid molecule (or other analyte). For example, the bead may be releasably coupled to the nucleic acid molecule, such as with a linker or with other methods described elsewhere herein. In some instances, the bead may be coupled to or attached to the nucleic acid molecule using click chemistry as described elsewhere herein. In some instances, the nucleic acid molecule (or other analyte) may be captured by the bead (e.g., in a polymer matrix). The first control bead may comprise any number of known sequences. For example, the first bead control bead may comprise at least about 1, 2, 3, 4, 5, 6, 7, 8, 9, 10, 15, 20, 25, 30, 35, 40, 45, 50, 55, 60, 65, 70, 75, 80, 85, 90, 95, 96, or 100 or more known sequences. Alternatively or in addition to, the first bead control bead may comprise at most about 100, 96, 95, 90, 85, 80, 75, 70, 65, 60, 55, 50, 45, 40, 35, 30, 25, 20, 15, 10, 9, 8, 7, 6, 5, 4, 3, 2, or 1 known sequences. The first control bead may comprise as many and/or any combination of different analytes (e.g., nucleic acid molecules). In an example, the first control bead may comprise a first DNA molecule comprising a first known sequence, a second DNA molecule comprising a second known sequence, and a RNA molecule comprising a third known sequence. The first control bead may comprise one or more additional analytes that do not comprise a known sequence. For example, such analytes may allow the first control bead to mimic single cell behavior.

A second control bead from the plurality of second control beads 912 may be another synthetic cell, such as described with respect to the first control bead. The second control bead may comprise a second set of one or more known sequences. For example, the bead may comprise a nucleic acid molecule (e.g., DNA, RNA, etc.) or other analyte (e.g., proteins, metabolite, nucleic acid molecule, etc.) comprising a known sequence. In some instances, the second control bead may be coupled to, or otherwise incorporate, the nucleic acid molecule (or other analyte). For example, the second control bead may be releasably coupled to the nucleic acid molecule, such as with a linker or with other methods described elsewhere herein. In some instances, the bead may be coupled to or attached to the nucleic acid molecule using click chemistry as described elsewhere herein. In some instances, the nucleic acid molecule (or other analyte) may be captured by the bead (e.g., in a polymer matrix). The second control bead may comprise any number of known sequences. For example, the second control bead may comprise at least about 1, 2, 3, 4, 5, 6, 7, 8, 9, 10, 15, 20, 25, 30, 35, 40, 45, 50, 55, 60, 65, 70, 75, 80, 85, 90, 95, 96, 100 or more known sequences. Alternatively or in addition to, the second control bead may comprise at most about 100, 96, 95, 90, 85, 80, 75, 70, 65, 60, 55, 50, 45, 40, 35, 30, 25, 20, 15, 10, 9, 8, 7, 6, 5, 4, 3, 2, or 1 known sequences. The second control bead may comprise as many and/or any combination of different analytes (e.g., nucleic acid molecules). In an example, the second control bead may comprise a first DNA molecule comprising a first known sequence, a second DNA molecule comprising a second known sequence, and a RNA molecule comprising a third known sequence. The second control bead may comprise one or more additional analytes that do not comprise a known sequence. For example, such analytes may allow the second control bead to mimic single cell behavior.

The first set of known sequences in the first control bead may be different from the second set of known sequences in the second control bead. For example, they may be mutually exclusive, such that none of the one or more known sequences in the second control bead overlaps with any of the one or more known sequences in the first control bead. In some instances, at least about 90%, 91%, 92%, 93%, 94%, 95%, 96%, 97%, 98%, 99%, 99.5%, 99.9% or more transcripts in the first set of known sequences may be separate from and independently classifiable from the second set of known sequences. Alternatively or in addition, at most about 99.9%, 99.5%, 99%, 98%, 97%, 96%, 95%, 94%, 93%, 92%, 91%, 90% or less transcripts in the first set of known sequences may be separate from and independently classifiable from the second set of known sequences. In other instances, there may be one or more overlapping known sequences.

In some instances, each set of known sequences may comprise a unique set of sequences. For example, the unique set of sequences may vary in length, concentration, and/or GC content.

In some instances, the known sequences may be synthetic. In other instances, the known sequences may be derived from a biological sample. Alternatively or in addition to, the known sequences may be a combination of synthetic and biologically derived sequences. In some instances, a first set of known sequences for the first control bead may be derived from a first species (e.g., purified genomic DNA from a human), and a second set of known sequences for the second control bead may be derived from a second species (e.g., purified genomic DNA from a mouse). In some instances, the sources of the known sequences used in the control beads may vary depending on the type of single cell application, type of parameter (e.g., UMI count purity, doublet rate, etc.) that is to be determined, and/or method of determination. For example, laboratories may use readily available, well-characterized cell lines to provide the known sequences as control. But using such controls may yield variable results depending on a variety of biological factors, and it may be difficult (or in some cases impossible) to calculate doublet rate or UMI purity. In another example, a first set of known sequences may be derived from the peripheral blood mononuclear cells (PBMCs) from a first human donor and a second set of known sequences may be derived from the PBMCs from a second human donor, and the single nucleotide polymorphisms (SNPs) may be analyzed to determine the doublet rate, but not the UMI purity. In another example, a first set of known sequences may be derived from the peripheral blood mononuclear cells (PBMCs) from a female human and a second set of known sequences may be derived from the PBMCs from a male human, and the Y chromosomes may be analyzed to determine the doublet rate, but not the UMI purity.

The systems and methods may comprise any ratio of the number or concentration of the plurality of first control beads 910 to the plurality of second control beads 912. For example, the ratio may be 1:1. Alternatively or in addition to, the ratio may be at least about 1:0.001, 1:0.01, 1:0.1, 1:0.2, 1:0.3, 1:0.4, 1:0.5, 1:0.6, 1:0.7, 1:0.8, 1:0.9, 1:1, 1:1.1, 1:1.2, 1:1.3, 1:1.4, 1:1.5, 1: 1.6, 1:1.7, 1:1.8, 1:1.9, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:1000 or more. Alternatively or in addition to, the ratio may be at most about 1:1000, 1;100, 1:90, 1:80, 1:70, 1:60, 1:50, 1:40, 1:30, 1:20, 1:10, 1:9, 1:8, 1:7, 1:6, 1:5, 1:4, 1:3, 1:2, 1:1.9, 1:1.8, 1:1.7, 1:1.6, 1:1.5, 1:1.4, 1:1.3, 1:1.2, 1: 1.1, 1:1, 1:0.9, 1:0.8, 1:0.7, 1:0.6, 1:0.5, 1:0.4, 1:0.3, 1:0.2, 1:0.1, 1:0.01, 1:0.001 or less.

Methods for generating a bead are described in detail elsewhere herein. A control bead may be generated as a gel bead, for example, encapsulating one or more nucleic acid molecules comprising the known sequences in the control bead. For example, nucleic acid molecules comprising the known sequences may be added into the starting reagent mix (e.g., instead of acrydite primers). The control bead may be configured to have smaller pore size, such as by increasing cross-linker concentration. For example, the pores may prevent DNA molecules, RNA molecules, or other analytes from escaping the control bead. In operation, the control beads may be packaged into correct buffers at a known concentration, and stored in single use aliquots. The control beads may be stored at ambient temperatures. The control beads may be stored at room temperatures. In some instances, the control beads may be stored at about −80 degrees Celsius (° C.). Alternatively or in addition to, the control beads may be stored at (or otherwise exposed to) temperatures of at most about 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4°° C., 3° C., 2°° C., 1° C., 0° C., −5° C., −10° C., −15° C., −20° C., −25° C., −30° C., −35° C., −40° C., −45° C., −50° C., −55° C., −60° C., −65° C., −70° C., −75° C., −80° C. or less. In some instances, the control beads may be stored at (or otherwise exposed to) temperatures of more than about 25° C.

Alternatively or in addition to, control beads of the present disclosure may be generated as non-gel beads, such as polystyrene beads, encapsulating one or more nucleic acid molecules comprising the known sequences.

In operation, as illustrated in FIG. 9, a plurality of first control beads 910 and a plurality of second control beads 912 may be introduced into a cell sample comprising a plurality of cells 908 to generate a mixed cell sample 902. Any number of each type of control bead may be introduced into the cell sample. For example, at least about 2, 3, 4, 5, 6, 7, 8, 9, 10, 20, 30, 40, 50, 60, 70, 80, 90, 100, 110, 120, 130, 140, 150, 200, 300, 400, 500, 600, 700, 800 or more control beads may be introduced. Alternatively or in addition to, at most about 800, 700, 600, 500, 400, 300, 200, 150, 140, 130, 120, 110, 100, 90, 80, 70, 60, 50, 40, 30, 20, 10, 9, 8, 7, 6, 5, 4, 3, 2, or 1 control bead may be introduced into the cell sample. Alternatively or in addition to, a ratio of a first concentration of first control beads and/or second control beads to a second concentration of cells in the mixed cell sample may be at least about 1:1000, 1;100, 1:90, 1:80, 1:70, 1:60, 1:50, 1:40, 1:30, 1:20, 1:10, 1:9, 1:8, 1:7, 1:6, 1:5, 1:4, 1:3, 1:2, 1:1.9, 1:1.8, 1:1.7, 1:1.6, 1:1.5, 1:1.4, 1:1.3, 1:1.2, 1:1.1, 1:1, 1:0.9, 1:0.8, 1:0.7, 1:0.6, 1:0.5, 1:0.4, 1:0.3, 1:0.2, 1:0.1, 1:0.01, 1:0.001 or more. Alternatively or in addition to, the ratio may be at most about 1:0.001, 1:0.01, 1:0.1, 1:0.2, 1:0.3, 1:0.4, 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:3, 1:4, 1:5 1:6, 1:7, 1:8, 1:9, 1:10, 1:20, 1:30, 1:40, 1:50, 1:60, 1:70, 1:80, 1:90, 1:100, 1:1000 or less. The mixed cell sample 902 may be partitioned with the set of barcode beads 904 to generate the plurality of partitions 906. Each partition in the plurality of partitions 906 may comprise a barcode bead and one of a cell, a first control bead, or a second control bead. For example, some partitions 916 may comprise a barcode bead and a cell, some partitions 918 may comprise a barcode bead and a first control bead, and some partitions 920 may comprise a barcode bead and a second control bead. Libraries may be created and sequenced from the plurality of partitions 906.

For example, in a first partition (e.g., 916), a first cell (e.g., 908) is co-partitioned with a first barcode bead (e.g., 914). The first barcode bead may comprise one or more barcode nucleic acid molecules, each individual barcode nucleic acid molecule (i) sharing a common, bead-specific, first barcode sequence and (ii) having a different unique molecular identifier (UMI). In some instances, the first cell may be lysed in the partition (e.g., via co-partitioning with a lysing agent) to release one or more analyte carriers. The one or more analyte carriers may comprise template nucleic acid molecules, such as DNA, RNA, and the like. In the partition, the barcode nucleic acid molecules can be released from the first barcode bead to hybridize to the analyte carriers. By way of example, where an analyte carrier is an mRNA molecule, a poly-T segment of the barcode nucleic acid molecule can hybridize to the poly-A tail of the mRNA molecule to generate a barcoded mRNA molecule. Each analyte carrier from the first cell may be hybridized to a different barcode nucleic acid molecule, thereby each being labeled by the first barcode sequence and a different UMI. For example, a barcoded template nucleic acid molecule may be generated.

In a second partition (e.g., 918), a first control bead (e.g., 910) is co-partitioned with a second barcode bead (e.g., 914). The second barcode bead may comprise one or more barcode nucleic acid molecules, each individual barcode nucleic acid molecule (i) sharing a common, bead-specific, second barcode sequence and (ii) having a different unique molecular identifier (UMI). The first control bead may comprise a plurality of analyte carriers. By way of example, the analyte carriers may comprise a first nucleic acid molecule with a first known sequence and a second nucleic acid molecule with a second known sequence. In some instances, the first control bead may be stimulated to release the first nucleic acid molecule and the second nucleic acid molecule from the first control bead. In the partition, the barcode nucleic acid molecules can be released from the second barcode bead to hybridize to the analyte carriers of the first control bead and generate barcoded nucleic acid molecules. Each analyte carrier from the first control bead may be hybridized to a different barcode nucleic acid molecule, thereby each being labeled by the second barcode sequence and a different UMI. For example, a first barcoded nucleic acid molecule and a second barcoded nucleic acid molecule may be generated. The first barcoded nucleic acid molecule may comprise the first known sequence, the second barcode sequence, and a UMI. The second barcoded nucleic acid molecule may comprise the second known sequence, the second barcode sequence, and a UMI.

In a third partition (e.g., 920), a second control bead (e.g., 912) is co-partitioned with a third barcode bead (e.g., 914). The third barcode bead may comprise one or more barcode nucleic acid molecules, each individual barcode nucleic acid molecule (i) sharing a common, bead-specific, third barcode sequence and (ii) having a different unique molecular identifier (UMI). The second control bead may comprise a plurality of analyte carriers. By way of example, the analyte carriers may comprise a third nucleic acid molecule with a third known sequence and a fourth nucleic acid molecule with a fourth known sequence. In some instances, the second control bead may be stimulated to release the third nucleic acid molecule and the fourth nucleic acid molecule from the second control bead. In the partition, the barcode nucleic acid molecules can be released from the third barcode bead to hybridize to the analyte carriers of the second control bead and generate barcoded nucleic acid molecules. Each analyte carrier from the second control bead may be hybridized to a different barcode nucleic acid molecule, thereby each being labeled by the third barcode sequence and a different UMI. For example, a third barcoded nucleic acid molecule and a fourth barcoded nucleic acid molecule may be generated. The third barcoded nucleic acid molecule may comprise the third known sequence, the third barcode sequence, and a UMI. The fourth barcoded nucleic acid molecule may comprise the fourth known sequence, the third barcode sequence, and a UMI.

The barcoded nucleic acid molecules from the plurality of partitions 906 may be sequenced. In the above example, from the sequencing results, the first barcode sequence may be identified to identify the first cell, the second barcode sequence may be identified to identify the first control bead, and the third barcode sequence may be identified to identify the second control bead. From the sequencing results, the first known sequence may be identified to identify the first nucleic acid molecule and the first control bead, the second known sequence may be identified to identify the second nucleic acid molecule and the first control bead, the third known sequence may be identified to identify the third nucleic acid molecule and the second control bead, and the fourth known sequence may be identified to identify the fourth nucleic acid molecule and the second control bead.

During the single cell process, each cell or control bead in the mixed cell sample 902 is barcoded by a unique bead-specific barcode sequence, such that each analyte having the same bead-specific barcode sequence may be attributed to the same cell or control bead. However, a doublet situation may arise where two or more cells and/or control beads are barcoded by the same bead-specific sequence. For example, where a first cell and a second cell are barcoded by the same bead-specific barcode sequence, two analytes that respectively originated from the first cell and the second cell, and having been barcoded by the same bead-specific barcode sequence, may incorrectly be attributed as having originated from the same cell. For example, a doublet situation may be caused by two or more beads in the set of beads 904 having the same bead-specific barcode sequence or where a partition co-partitions one bead with two or more cells and/or control beads (which is more likely). An example of the latter situation is illustrated in partition 920. Beneficially, the systems and methods described herein may measure doublet rate. Doublet rate may be measured by comparing the barcode sequence on the known sequences in the first control bead (e.g., first known sequence, second known sequence, etc.) with the barcode sequence on the known sequences in the second control bead (e.g., third known sequence, fourth known sequence, etc.). Any overlap may be indicative of doublets. Doublet rate may be measured based at least in part on the number of overlapping instances and the concentration of the first control beads 910 and/or the second control beads 912 in the mixed cell sample 902. For clarity, for the purpose of measuring or investigating doublet rate, the presence of UMIs is not required. That is, the barcode bead need not contain different UMIs on individual barcode nucleic acid molecules (i.e., their presence or absence is optional). For example, a barcode bead used in the systems and methods described herein may comprise one or more barcode nucleic acid molecules, each individual barcode nucleic acid molecule sharing a common, bead-specific, barcode sequence.

In some instances, each analyte may be barcoded by a unique molecular identifier. Ideally, during the single cell process, each analyte in a cell or control bead is barcoded by a truly unique molecular identifier, such that each analyte has a different unique molecular identifier. Beneficially, identical copies of analytes arising from distinct cells may be distinguished from the identical copies arising from amplification (e.g., PCR). Even following any subsequent amplification of the contents of a given partition, the number of different UMIs can be indicative of the quantity of the analyte originating from a given partition, and thus from the analyte carrier (e.g., cell). Such UMI counts may be used to compare, for example, the number of copies of each transcript in different cell types or conditions. However, there may be errors in a UMI count when, for example, foreign UMIs are introduced during the single cell process. For example, a UMI sequence may be changed when there are nucleotide substitutions during PCR or nucleotide miscalling, or insertions or deletions (indels) during sequencing. For example, such changes may be caused by recombination events during PCR that create chimeric sequences that can change an original UMI sequence. Beneficially, the systems and methods described herein may measure UMI purity. Beneficially, the systems and methods described herein may measure both doublet rate and UMI purity in the same assay. In the above example, UMI purity may be measured by comparing the UMI count for the first control bead to an expected UMI count based on the known sequences in the first control bead (e.g., first known sequence, second known sequence, etc.). Alternatively or in addition to, UMI purity may be measured by comparing the UMI count for the second control bead to an expected UMI count based on the known sequences in the second control bead (e.g., third known sequence, fourth known sequence, etc.). UMI purity may be measured based at least in part on the concentration of the first control beads 910 and/or the second control beads 912 in the mixed cell sample 902.

As described elsewhere herein, the systems and methods may use any number of types of control beads. For example, there may be at least about 2, 3, 4, 5, 6, 7, 8, 9, 10, 15, 20, or more sets of control beads. Alternatively, there may be at most about 20, 15, 10, 9, 8, 7, 6, 5, 4, 3, 2, or 1 set of control beads. Such multiple sets of control beads may be introduced into the cell sample at any ratio.

The systems and methods described herein may be used to perform quality control on any single cell assay or process thereof, such as a partitioning process, barcoding process, amplification process, library creation and sequencing process, and other processes (or combinations thereof).

In some instances, a control bead may be configured to mimic different analytes. For example, a control bead of the present disclosure may be configured to mimic cell beads containing cells for analyzing single cell applications utilizing cell beads. Cell beads are described in detail elsewhere herein, such as with respect to FIG. 1. For example the control bead may be about 54 um in diameter to mimic a cell bead. Alternatively or in addition to, the control bead may have a diameter from at least about 10, 11, 12, 13, 14, 15, 16, 17, 18, 19, 20, 21, 22, 23, 24, 25, 26, 27, 28, 29, 30, 35, 40, 45, 50, 55, 60, 65 μm or greater. Alternatively or in addition to, the control bead may have a diameter of at most about 65, 60, 55, 50, 45, 40, 35, 30, 29, 28, 27, 26, 25, 24, 23, 22, 21, 20, 19, 18, 17, 16, 15, 14, 13, 12, 11, 10 μm or less. In some instances, the size (e.g., diameter) of the control bead may deviate from an average size (e.g., diameter) of a cell bead in a mixed cell bead sample (e.g., mixing a plurality of cell beads with the control bead(s)) within at least about 90%, 80%, 70%, 60%, 50%, 40%, 30%, 25%, 20%, 15%, 10%, 9%, 8%, 7%, 6%, 5%, 4%, 3%, 2%, 1%, or lower. Alternatively or in addition to, the control bead may have other physical characteristics or properties that mimic a cell bead, such as density, mass, shape, and the like. The control bead may have other characteristics or properties, such as chemical properties, that mimic a cell bead, such as conductivity, hardness, size, shape, density, hydrophobicity, and/or interactive properties with aqueous or non-aqueous solutions, and the like. In operation, the control beads may be introduced to a cell bead sample, and the mixed cell bead sample may be subjected to the single cell application or assay. The control beads, or known sequences thereof, may be tracked during or after the single cell application or assay for analysis.

In some instances, a control bead may be configured to mimic, or comprise, a chromatin. For example, nucleic acid fragments may be generated (e.g., synthetic or biologically derived) to have defined protein binding sites as well as open stretches. The control bead may comprise a protein-DNA molecule complex, wherein the DNA molecule in the complex comprises a known sequence. The known sequence may comprise the defined protein binding sites. The control bead may be mixed with other analytes comprising protein-DNA molecule complexes for use in single cell applications, such as an Assay for Transposase Accessible Chromatin (ATAC-seq). The control bead may be configured to mimic, or comprise, any other analyte (e.g., cell, cell bead, chromatin, nucleic acid molecule, protein, another molecule, a complex, etc.) for use as controls in other single cell applications. The control beads, or known sequences thereof, may be tracked during or after the single cell application or assay for analysis.

### Kits

The present disclosure provides kits for use with methods and systems described herein. A kit can include a set of control beads. The set of control beads may be any set of control beads described elsewhere herein. For example, the set of control beads may be configured to mimic cells, cell beads, or other analytes. The kit may include an index comprising the set of known nucleic acid sequences in the set of control beads. For example, the index may be list of the known nucleic acid sequences. In some instances, the index may comprise the concentration of each known sequence. In some instances, the index may comprise the concentration of the set of control beads in a buffer. In some instances, the index may comprise the concentration of any other reagents included in the kit. The kit can include reagents and buffers necessary for performing the methods described herein. For example, the kit can include reagents and buffers for sample preparation for a sequencing assay and/or reagents and buffers for performing one or more sequencing assays described herein.

The kit may comprise any number of sets of control beads, such as described elsewhere herein. For example, the kit may comprise at least about 2, 3, 4, 5, 6, 7, 8, 9, 10, 20, 30, 40, 50 or more sets of control beads. Alternatively or in addition to, the kit may comprise at most about 50, 40, 30, 20, 10, 9, 8, 7, 6, 5, 4, 3, 2, or 1 set of control beads. Such set(s) (or subset(s)) of control beads may be used in a kit at any ratio (e.g., in a 1:2 ratio). The index may comprise the sets of known nucleic acid sequences for each set of control beads included in the kit. The index may comprise the concentration of each known sequence. The index may comprise the concentration of each set of control beads in a respective buffer.

The kit may comprise instructions detailing one or more processes of the systems and methods described herein. The kit can include a carrier, package, or container that may be compartmentalized to receive one or more containers, such as vials, tubes, and the like, each of the container(s) comprising one of the separate elements, such as the nucleic acid probes and buffers, to be used in a method described herein. Suitable containers include, for example, bottles, vials, syringes, and test tubes. The containers can be formed from a variety of materials such as glass or plastic. The kit may include any combination of the above. A kit may include all of the above. The articles of manufacture provided herein contain packaging materials. Examples of packaging materials include, but are not limited to, bottles, tubes, bags, containers, or bottles. A kit can include labels listing contents of the kit and/or instructions for use, and package inserts with instructions for use. A set of instructions can also be included. The instructions may be in physical or digital format (e.g., instructions that may be included in a pamphlet or stored in computer memory).

### Systems and Methods for Sample Compartmentalization

In an aspect, the systems and methods described herein provide for the compartmentalization, depositing, or partitioning of one or more particles (e.g., analyte carriers, macromolecular constituents of analyte carriers, beads, reagents, etc.) into discrete compartments or partitions (referred to interchangeably herein as partitions), where each partition maintains separation of its own contents from the contents of other partitions. The partition can be a droplet in an emulsion. A partition may comprise one or more other partitions.

A partition may include one or more particles. A partition may include one or more types of particles. For example, a partition of the present disclosure may comprise one or more analyte carriers and/or macromolecular constituents thereof. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be an analyte carrier and/or one or more of its macromolecular constituents encased inside of a gel or polymer matrix, such as via polymerization of a droplet containing the analyte carrier and precursors capable of being polymerized or gelled. Unique identifiers, such as barcodes, may be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms may also be employed in the partitioning of individual analyte carriers, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual particles to discrete partitions may in one non-limiting example be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of analyte carriers per partition, number of beads per partition, etc.). For example, partition occupancy can be controlled by providing the aqueous stream at a certain concentration and/or flow rate of particles. To generate single analyte carrier partitions, the relative flow rates of the immiscible fluids can be selected such that, on average, the partitions may contain less than one analyte carrier per partition in order to ensure that those partitions that are occupied are primarily singly occupied. In some cases, partitions among a plurality of partitions may contain at most one analyte carrier (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

FIG. 1 shows an example of a microfluidic channel structure 100 for partitioning individual analyte carriers. The channel structure 100 can include channel segments 102, 104, 106 and 108 communicating at a channel junction 110. In operation, a first aqueous fluid 112 that includes suspended analyte carriers (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual analyte carrier 114 (such as droplets 118). A discrete droplet generated may include more than one individual analyte carrier 114 (not shown in FIG. 1). A discrete droplet may contain no analyte carrier 114 (such as droplet 120). Each discrete partition may maintain separation of its own contents (e.g., individual analyte carrier 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., analyte carriers, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more analyte carriers 114, and (2) unoccupied droplets 120, not containing any analyte carriers 114. Occupied droplets 118 may comprise singly occupied droplets (having one analyte carrier) and multiply occupied droplets (having more than one analyte carrier). As described elsewhere herein, in some cases, the majority of occupied partitions can include no more than one analyte carrier per occupied partition and some of the generated partitions can be unoccupied (of any analyte carrier). In some cases, though, some of the occupied partitions may include more than one analyte carrier. In some cases, the partitioning process may be controlled such that fewer than about 25% of the occupied partitions contain more than one analyte carrier, and in many cases, fewer than about 20% of the occupied partitions have more than one analyte carrier, while in some cases, fewer than about 10% or even fewer than about 5% of the occupied partitions include more than one analyte carrier per partition.

In some cases, it may be desirable to minimize the creation of excessive numbers of empty partitions, such as to reduce costs and/or increase efficiency. While this minimization may be achieved by providing a sufficient number of analyte carriers (e.g., analyte carriers 114) at the partitioning junction 110, such as to ensure that at least one analyte carrier is encapsulated in a partition, the Poissonian distribution may expectedly increase the number of partitions that include multiple analyte carriers. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the analyte carriers (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

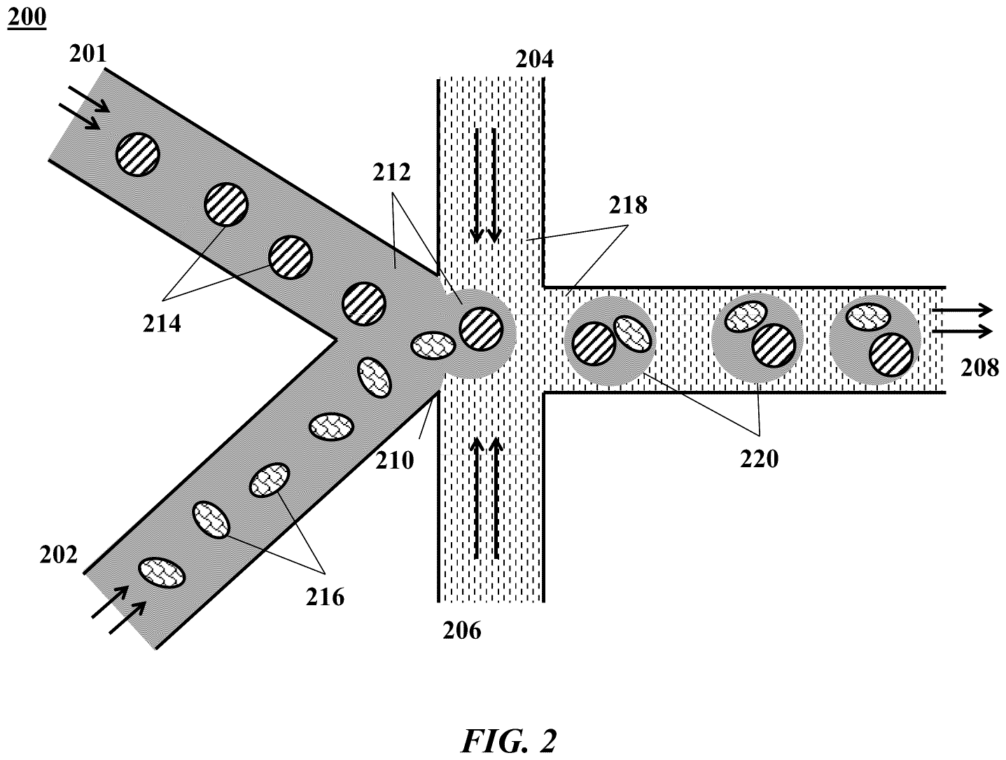

As will be appreciated, the above-described occupancy rates are also applicable to partitions that include both analyte carriers and additional reagents, including, but not limited to, microcapsules or beads (e.g., gel beads) carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and an analyte carrier.

In another aspect, in addition to or as an alternative to droplet based partitioning, analyte carriers may be encapsulated within a microcapsule that comprises an outer shell, layer or porous matrix in which is entrained one or more individual analyte carriers or small groups of analyte carriers. The microcapsule may include other reagents. Encapsulation of analyte carriers may be performed by a variety of processes. Such processes may combine an aqueous fluid containing the analyte carriers with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising analyte carriers may be performed by a variety of methods. For example, air knife droplet or aerosol generators may be used to dispense droplets of precursor fluids into gelling solutions in order to form microcapsules that include individual analyte carriers or small groups of analyte carriers. Likewise, membrane based encapsulation systems may be used to generate microcapsules comprising encapsulated analyte carriers as described herein. Microfluidic systems of the present disclosure, such as that shown in FIG. 1, may be readily used in encapsulating cells as described herein. In particular, and with reference to FIG. 1, the aqueous fluid 112 comprising (i) the analyte carriers 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained analyte carriers. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca²⁺ions), can be used as an encapsulation process using the described processes. Likewise, agarose droplets may also be transformed into capsules through temperature based gelling (e.g., upon cooling, etc.).

In some cases, encapsulated analyte carriers can be selectively releasable from the microcapsule, such as through passage of time or upon application of a particular stimulus, that degrades the microcapsule sufficiently to allow the analyte carriers (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes. The analyte carrier can be subjected to other conditions sufficient to polymerize or gel

the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the analyte carrier. The polymer or gel may be diffusively permeable to chemical or biochemical reagents. The polymer or gel may be diffusively impermeable to macromolecular constituents of the analyte carrier. In this manner, the polymer or gel may act to allow the analyte carrier to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the analyte carrier may be surrounded by polyacrylamide strands linked together by disulfide bridges. In this manner, the analyte carrier may be encased inside of or comprise a gel or matrix (e.g., polymer matrix) to form a “cell bead.” A cell bead can contain analyte carriers (e.g., a cell) or macromolecular constituents (e.g., RNA, DNA, proteins, etc.) of analyte carriers. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing analyte carriers and cell beads (and/or droplets or other partitions) containing macromolecular constituents of analyte carriers.

Encapsulated analyte carriers can provide certain potential advantages of being more storable and more portable than droplet-based partitioned analyte carriers. Furthermore, in some cases, it may be desirable to allow analyte carriers to incubate for a select period of time before analysis, such as in order to characterize changes in such analyte carriers over time, either in the presence or absence of different stimuli. In such cases, encapsulation may allow for longer incubation than partitioning in emulsion droplets, although in some cases, droplet partitioned analyte carriers may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of analyte carriers may constitute the partitioning of the analyte carriers into which other reagents are co-partitioned. Alternatively or in addition, encapsulated analyte carriers may be readily deposited into other partitions (e.g., droplets) as described above.

### Beads

A partition may comprise one or more unique identifiers, such as barcodes. Barcodes may be previously, subsequently or concurrently delivered to the partitions that hold the compartmentalized or partitioned analyte carrier. For example, barcodes may be injected into droplets previous to, subsequent to, or concurrently with droplet generation. The delivery of the barcodes to a particular partition allows for the later attribution of the characteristics of the individual analyte carrier to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of analyte carriers 216 along the channel segment 202 into junction 210. The plurality of analyte carriers 216 may be sourced from a suspension of analyte carriers. For example, the channel segment 202 may be connected to a reservoir comprising an aqueous suspension of analyte carriers 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested.

As an alternative, the channel segments 201 and 202 may meet at another junction upstream of the junction 210. At such junction, beads and analyte carriers may form a mixture that is directed along another channel to the junction 210 to yield droplets 220. The mixture May provide the beads and analyte carriers in an alternating fashion, such that, for example, a droplet comprises a single bead and a single analyte carrier.

Beads, analyte carriers and droplets may flow along channels at substantially regular flow profiles (e.g., at regular flow rates). Such regular flow profiles may permit a droplet to include a single bead and a single analyte carrier. Such regular flow profiles may permit the droplets to have an occupancy (e.g., droplets having beads and analyte carriers) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 218 can comprise an oil, such as a fluorinated oil, that includes a fluorosurfactant for stabilizing the resulting droplets, for example, inhibiting subsequent coalescence of the resulting droplets 220.

A discrete droplet that is generated may include an individual analyte carrier 216. A discrete droplet that is generated may include a barcode or other reagent carrying bead 214. A discrete droplet generated may include both an individual analyte carrier and a barcode carrying bead, such as droplets 220. In some instances, a discrete droplet may include more than one individual analyte carrier or no analyte carrier. In some instances, a discrete droplet may include more than one bead or no bead. A discrete droplet may be unoccupied (e.g., no beads, no analyte carriers).

Beneficially, a discrete droplet partitioning an analyte carrier and a barcode carrying bead may effectively allow the attribution of the barcode to macromolecular constituents of the analyte carrier within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead. In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an RI primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

FIG. 8 illustrates an example of a barcode carrying bead. A nucleic acid molecule 802, such as an oligonucleotide, can be coupled to a bead 804 by a releasable linkage 806, such as, for example, a disulfide linker. The same bead 804 may be coupled (e.g., via releasable linkage) to one or more other nucleic acid molecules 818, 820. The nucleic acid molecule 802 may be or comprise a barcode. As noted elsewhere herein, the structure of the barcode may comprise a number of sequence elements. The nucleic acid molecule 802 may comprise a functional sequence 808 that may be used in subsequent processing. For example, the functional sequence 808 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 802 may comprise a barcode sequence 810 for use in barcoding the sample (e.g., DNA, RNA, protein, etc.). In some cases, the barcode sequence 810 can be bead-specific such that the barcode sequence 810 is common to all nucleic acid molecules (e.g., including nucleic acid molecule 802) coupled to the same bead 804. Alternatively or in addition, the barcode sequence 810 can be partition-specific such that the barcode sequence 810 is common to all nucleic acid molecules coupled to one or more beads that are partitioned into the same partition. The nucleic acid molecule 802 may comprise a specific priming sequence 812, such as an mRNA specific priming sequence (e.g., poly-T sequence), a targeted priming sequence, and/or a random priming sequence. The nucleic acid molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid molecules (e.g., 802, 818, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting mRNA molecule that was captured, in order to allow quantification of the number of original expressed RNA. As will be appreciated, although FIG. 8 shows three nucleic acid molecules 802, 818, 820 coupled to the surface of the bead 804, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 808, 810, 812, etc.) and variable or unique sequence segments (e.g., 816) between different individual nucleic acid molecules coupled to the same bead.

In operation, an analyte carrier (e.g., cell, DNA, RNA, etc.) can be co-partitioned along with a barcode bearing bead 804. The barcoded nucleic acid molecules 802, 818, 820 can be released from the bead 804 in the partition. By way of example, in the context of analyzing sample RNA, the poly-T segment (e.g., 812) of one of the released nucleic acid molecules (e.g., 802) can hybridize to the poly-A tail of an mRNA molecule. Reverse transcription may result in a cDNA transcript of the mRNA, but which transcript includes each of the sequence segments 808, 810, 816 of the nucleic acid molecule 802. Because the nucleic acid molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810. However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the analyte carrier (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) comprising barcoded nucleic acid molecules (e.g., oligonucleotides) within a single partition. Accordingly, as noted above, the flow characteristics of the analyte carrier and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of microcapsules from each source, while ensuring a given pairing or combination of such beads into a partition with a given number of analyte carriers (e.g., one analyte carrier and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100pL, 50 pL, 20 pL, 10 pL, 1 pL, or less. Where co-partitioned with microcapsules, it will be appreciated that the sample fluid volume, e.g., including co-partitioned analyte carriers and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

The compositions, methods and kits described herein can comprise a plurality of partitions, wherein one or more partitions of the plurality of partitions may comprise one or more barcode-containing beads, one or more synthetic cells (or control beads), or any combination thereof. As described herein, control beads can be used as control elements or internal standards in the processing of a biological sample (e.g., single cell analysis). A partition may comprise one or more different beads. Such a bead may be a barcode-containing bead or a control bead such as a synthetic cell. Such a bead as may be a control bead (e.g., a synthetic cell).

A bead may comprise various molecules. Such molecules may be nucleic acid molecules or polypeptides. A bead may be a control bead. A control bead may be coupled to, attached to, or otherwise associated with a molecule using conjugation chemistry. Conjugation chemistry as described herein refers to any chemical reaction that links, couples, or attaches a first molecule with a second molecule. Conjugation chemistry may comprise bioconjugation chemistry and click chemistry. Conjugation chemistry may comprise biological interactions (e.g., biotin/strepdavidin interactions) and/or bioorthogonal reactions.

In addition to using click chemistry, a molecule (e.g., a nucleic acid molecule) can be attached to a bead (e.g., a gel bead) using various other bioconjugation or coupling methods. Such bioconjugation methods can include various conjugation strategies and functional group modifications such as mesylate formation, sulfur alkylation, NHS ester formation, carbamate formation, carbonate formation, amide bond formation, or any combination thereof. Such strategies and functional group modifications can be used for various reaction types such nucleophilic and/or electrophilic substitution reaction, nucleophilic and/or electrophilic addition reaction, and other suitable reaction types. In some cases, activated carboxylic acids can react with nucleophiles such as amines. In some cases, the carboxylic acid can be attached to a bead (e.g., a gel bead, control bead, etc.) and the nucleophilic group such as an amine can be attached to a molecule (e.g., a nucleic acid molecule) to be attached to said bead. Such amide bond formation reactions can include EDC/NHS (e.g., via 1-ethyl-3-(3-dimethyl-aminopropyl)carbodiimide (EDC) and N-hydroxysuccinimide (NHS) or 4-(4,6-dimethoxy-1,3,5-triazin-2-yl)-4-methylmorpholinium chloride (DMTMM)) mediated coupling reactions, wherein an activated ester (e.g., an NHS ester attached to a bead surface) can react with an amine (e.g., an amine of a nucleic acid molecule) to form an amide bond, thereby attaching said molecule (e.g., a nucleic acid molecule) to said bead (e.g., a gel bead). Any other suitable bioconjugation reactions can be used to attach a molecule to a bead.

Coupling or attachment of nucleic acid molecules to barcode beads and/or control beads (e.g., synthetic cells) may be performed using click chemistry. Click chemistry may comprise any type of click reaction suitable for the functionalization of synthetic cells. Examples of click chemistry reactions (or short “click reactions”) that may be used in combination with the herein described methods and compositions include, but are not limited to, transition-metal catalyzed or strain-promoted azide-alkyne cycloadditions (e.g., Huisgen azide-alkyne 1,3-dipolar cycloaddition, copper-catalyzed azide-alkyne cycloaddition (CuAAC), strain-promoted alkyne-azide cycloaddition, and/or ruthenium-catalyzed azide-alkyne cycloaddition (RuAAC)), Diels-Alder reactions such as inverse-electron demand Diels-Alder reaction (e.g., tetrazine-trans-cyclooctene reactions), or photo-click reactions (e.g., alkene-tetrazole photoreactions).

A bead may be attached to one or more sets of nucleic acid molecules using such click chemistry. Such a bead may be a control bead. A control bead may comprise (e.g., may be functionalized with) a first functional group. The first functional group may be a first reactant for a click reaction. The one or more sets of nucleic acid molecules that may be attached to a control bead may comprise a second functional group. The first functional group may be a second reactant for a click reaction. The click reaction is a copper-catalyzed azide-alkyne cycloaddition reaction, an inverse-electron demand Diels-Alder reaction, or an avidin-biotin interaction. In some instances, click reaction is a copper-catalyzed azide-alkyne cycloaddition reaction comprising an azide-functionalized nucleic acid molecule (e.g., a nucleic acid molecule comprising a known sequence) and an alkyne-functionalized bead (e.g., a control bead).

The first functional group may react with a second functional group in a click reaction. In some instances, a nucleic acid molecule comprising a known sequence may be attached to a control bead by reacting said first functional group with said second functional group. The first functional group is an alkyne, a trans-cyclooctene, or an avidin, or any combination thereof. In various instances, the first functional group is an alkyne. The second functional group is an azide, a tetrazine, or a biotin, or a combination thereof. In various instances, the second functional group is an azide.

A nucleic acid molecule may be releasably attached to a bead (e.g., a control bead). A nucleic acid molecule may be releasably attached to a bead via a releasable linkage. A nucleic acid molecule may be releasably attached to a bead via a cleavable sequence. Such a cleavable sequence may be a nucleotide sequence and/or an amino acid sequence. A cleavable sequence may release a nucleic acid molecule upon application of a particular stimulus. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators), pH-mediated, etc.), mechanical stimuli, or a combination thereof.

A nucleic acid molecule (or any other analyte) may be attached to a bead using click reactions as described herein. Such click reactions may comprise various reaction parameters. Such reaction parameters may include reaction temperature, reaction time, and any additional reagents present in the reaction solution. The reaction parameters may depend on the type of click reaction to be conducted. For example, a copper-catalyzed azide-alkyne cycloaddition reaction may comprise one or more different reaction parameters than an avidin-biotin interaction.

A bead may be functionalized to comprise a first functional group providing reactivity for certain reactions (e.g., a click reaction with a functionalized nucleic acid molecule). A nucleic acid molecule to be attached to a bead may be functionalized to comprise a second functional group providing reactivity for certain reactions (e.g., a click reaction with a functionalized bead). A nucleic acid molecule may be functionalized to comprise an azide moiety.

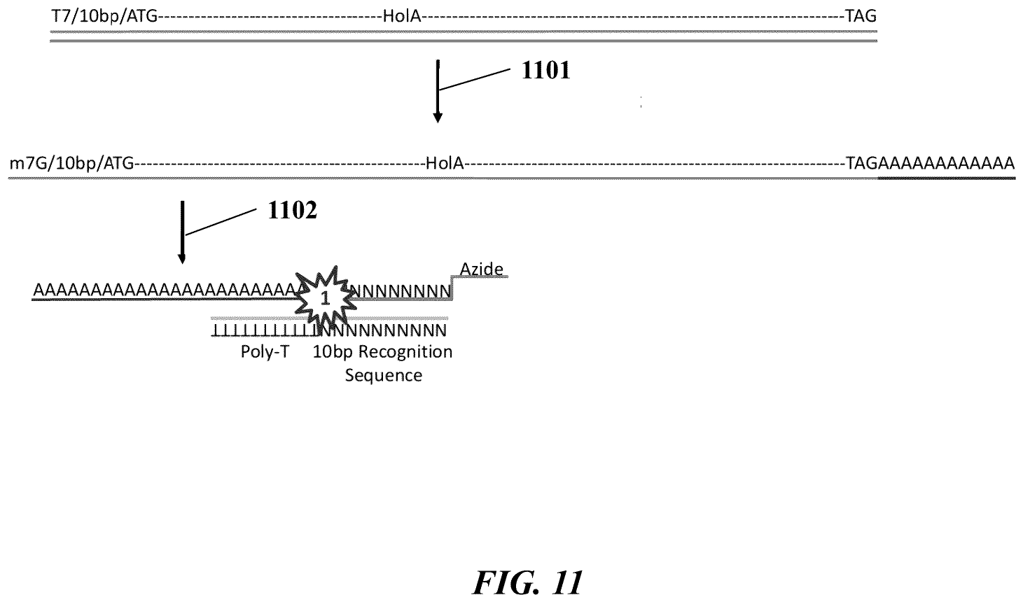

In operation, as illustrated in step 1102 of FIG. 11 (see also EXAMPLE 1), a ligation reaction for modifying a nucleic acid molecule to comprise an azide functional groups can comprise a solution (having a concentration of e.g., 1-100 μM, 10-50 μM, or 5-20 μM) of synthesized nucleic acid (e.g., RNA), splint or bridge oligonucleotides (e.g., in a concentration of 1-100 μM, 10-50 μM, or 5-20 μM), an azide oligonucleotide (e.g., in a concentration of 1-100 μM, 10-50 μM, or 5-20 μM), and ligase, wherein the ligase may be present in a concentration of about 10-100 μg/μL, 1-100 μM, 10-50 μM, or 5-20 μM. The reaction solution may be further diluted and/or adjusted to a specific volume by adding ligation buffer. As an example, a ligation reaction for modifying a RNA molecule to comprise an azide functional groups can comprise 10 uM synthesized RNA, 10 uM splint, 40 uM azide oligo, and 100 U/uL or 50 ug/uL ligase, ligation buffer can be added up to a certain volume (e.g., 250 uL). The reaction may be incubated at a certain temperature (e.g., about 16 degrees (° C.) for a certain period of time (e.g., 1-3 hours). Addition of EDTA (e.g., 1-10 μL or an amount to obtain a certain concentration) may be added to the reaction solution to stop (or quench) the reaction. Quantitation of the ligation reaction may be performed using various methods including the use of fluorescent dyes (e.g., AlexaFluor488, AlexaFluor647 etc.) and/or instruments such as Nanodrop, etc. that allow high-throughput screening of various reaction conditions.

A bead comprising a first functional group may be attached to a nucleic acid molecule comprising a second functional group. The bead may be attached to the nucleic acid molecule via bioconjugation chemistry. Bioconjugation methods can include various conjugation strategies and functional group modifications such as mesylate formation, sulfur alkylation, NHS ester formation, carbamate formation, carbonate formation, amide bond formation, or any combination thereof. Such strategies and functional group modifications can be used for various reaction types such nucleophilic and/or electrophilic substitution reaction, nucleophilic and/or electrophilic addition reaction, and other suitable reaction types. In some cases, activated carboxylic acids can react with nucleophiles such as amines. In some cases, the carboxylic acid can be attached to a bead (e.g., a gel bead, control bead, etc.) and the nucleophilic group such as an amine can be attached to a molecule (e.g., a nucleic acid molecule) to be attached to said bead. Such amide bond formation reactions can include EDC/NHS (e.g., via 1-ethyl-3-(3-dimethyl-aminopropyl)carbodiimide (EDC) and N-hydroxysuccinimide (NHS) or 4-(4,6-dimethoxy-1,3,5-triazin-2-yl)-4-methylmorpholinium chloride (DMTMM)) mediated coupling reactions, wherein an activated ester (e.g., an NHS ester attached to a bead surface) can react with an amine (e.g., an amine of a nucleic acid molecule) to form an amide bond, thereby attaching said molecule (e.g., a nucleic acid molecule) to said bead (e.g., a gel bead). Any other suitable bioconjugation reactions can be used to attach a molecule to a bead.

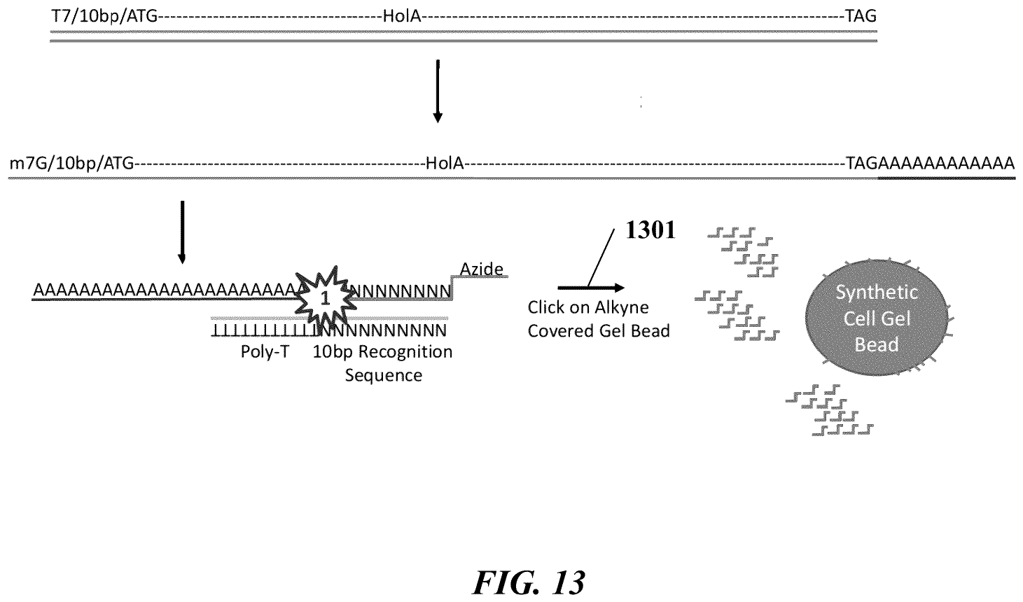

A nucleic acid molecule, or a modified derivative thereof, can be attached to a bead (e.g., a gel bead) using click chemistry. In operation, as illustrated in step 1301 of FIG. 13 (see also EXAMPLE 2), a nucleic acid molecule modified to comprise an azide moiety may be attached to a control bead via click chemistry. Such reactions may involve one or more additional reagents and/or buffer solutions. Such additional reagents may include catalysts. Such a catalyst may comprise one or more transition metals (e.g., copper) and one or more ligands (e.g., THTPA). The click reaction between an azide-functionalized nucleic acid molecule and an alkyne-functionalized control bead may be performed in a final reaction solution is which the click reaction is performed may have a certain volume and certain final concentrations of reactions. For example, a final reaction mixture for a copper-catalyzed azide-alkyne click reaction can comprise between about 100-1000 μL (e.g., 300 μL) of final volume, between about 0.5-5 mM (e.g., about 1.25 mM) THPTA ligand, between about 0.1-0.75 (e.g., about 0.25 mM) CuAcO₄, between about 1-10 mM (e.g., about 5.0 mM) sodium ascorbate and between about 0.01-0.1 mM (e.g., 0.04 mM) ligated (e.g., azide-functionalized) RNA, and between about 1-50 μM (e.g., 20 uM) gel beads such as control beads. As an example, a modified nucleic acid molecule comprising an azide moiety may be attached to an alkyne-functionalized control bead using the ligand THPTA at 50 mM, 10 mM copper acetate, 100 mM sodium ascorbate, 0.16 mM ligated RNA, and 20 uM gel beads/synthetic cells. Such a click reaction may be performed at room temperature. Reactions may be stopped or quenched using EDTA, for example.

Any bead described herein may be attached to a nucleic acid molecule using conjugation chemistry (e.g., click chemistry). The bead to be functionalized may be a control bead (e.g., synthetic cell) as described herein, and as such, may be used as a quality control element in various cell analyses processes, e.g., single-cell analyses of biological samples. A control bead may comprise (e.g., be functionalized with) one or more molecules. Such molecules may be the same molecules or may be different molecules. The molecule that a control bead may be functionalized with may be a nucleic acid molecule (e.g., RNA such as mRNA). Such a nucleic acid molecule may comprise a known sequence. A control bead may comprise hundreds, thousands, or millions of nucleic acid molecules. Such nucleic acid molecules may have an identical nucleic acid sequence or a different nucleic acid sequence. A control bead may comprise at least two different nucleic acid molecules. A control bead may comprise at least five different nucleic acid molecules. A control bead may comprise at least ten different nucleic acid molecules. A control bead may comprise at least twenty different nucleic acid molecules. A control bead may be functionalized with at least fifty different nucleic acid molecules. A control bead may be functionalized with at least a hundred different nucleic acid molecules. A control bead may be functionalized with about 1 to about 10 different nucleic acid molecules. A control bead may be functionalized with about 10 to about 25 different nucleic acid molecules. A control bead may be functionalized with about 20 to about 50 different nucleic acid molecules. A control bead may be functionalized with about 25 to about 75 different nucleic acid molecules. A control bead may be functionalized with about 50 to about 100 different nucleic acid molecules. A control bead may be functionalized with about 75 to about 150 different nucleic acid molecules. A control bead may be functionalized with about 100 to about 500 different nucleic acid molecules. A control bead may be functionalized with about 250 to about 1000 different nucleic acid molecules. A control bead may be functionalized with about 96 different nucleic acid molecules.

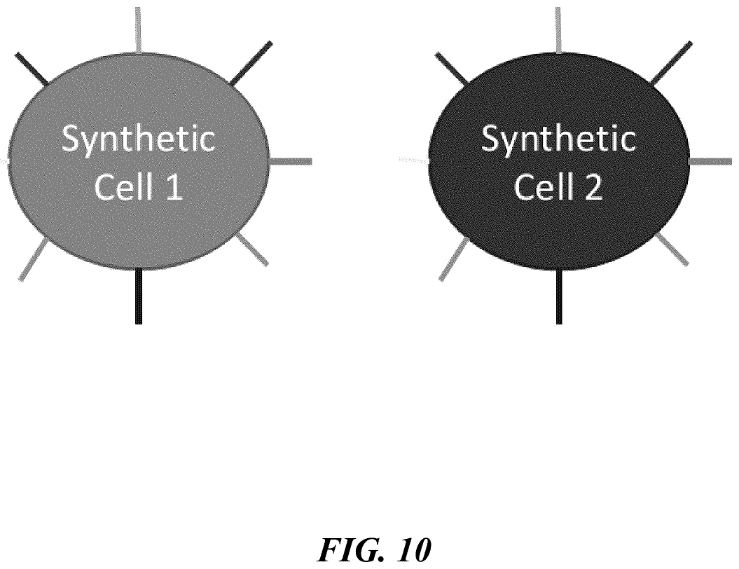

A control bead may comprise (e.g., may be functionalized with) two or more different nucleic acid sequences in two or more specific ratios (see e.g., FIG. 10, illustrating that one or more synthetic cells can each be functionalized with one or more different nucleic acid molecules). For example, a control bead may be functionalized with two different nucleic acid molecules (e.g., nucleic acid molecules comprising different nucleic acid sequences or are otherwise differently modified, such as those comprising different modifications at their 3′ and/or 5′ termini), wherein the bead may be functionalized with the two different nucleic acid molecules in a specific ratio. Such rations can vary widely, depending on application of the control bead or synthetic cell. As another example, a first control bead may be functionalized with 96 different nucleic acid sequences (e.g., genes), and a second control bead may be functionalized with another 96 different nucleic acid sequences (e.g., genes), wherein the 96 different nucleic acid sequences of the first control bead are different (e.g., comprise different nucleic acid sequences) compared to the 96 different nucleic acid sequences (e.g., genes) of the second control bead. Such first and second control beads may be used as quality control elements in cell analysis kits.

A control bead may comprise a first nucleic acid molecule and a second nucleic acid molecule. The first nucleic acid molecule and the second nucleic acid molecule may be attached to the control bead in a specific ratio. The ratio of the first nucleic acid molecule to the second nucleic acid molecule may be between about 10⁻³ to about 10³. The ratio of the first nucleic acid molecule to the second nucleic acid molecule may be between about 10⁻² to about 10⁻². The ratio of the first nucleic acid molecule to the second nucleic acid molecule may be between about 10⁻¹ to about 10. The ratio of the first nucleic acid molecule to the second nucleic acid molecule may be between about 1 to about 5. The ratio of the first nucleic acid molecule to the second nucleic acid molecule may be at least about 10⁻¹. The ratio of the first nucleic acid molecule to the second nucleic acid molecule may be at least about 10⁻¹. The ratio of the first nucleic acid molecule to the second nucleic acid molecule may be at least about 0.5. The ratio of the first nucleic acid molecule to the second nucleic acid molecule may be at least about 1. The ratio of the first nucleic acid molecule to the second nucleic acid molecule may be at least about 5. The ratio of the first nucleic acid molecule to the second nucleic acid molecule may be at least about 10. The ratio of the first nucleic acid molecule to the second nucleic acid molecule may be at least about 10².

The compositions, methods and kits of the present disclosure may comprise a first control bead and a second control bead. The first control bead and the second control bead may be used and/or be present in a specific ratio. The ratio of the first control bead to the second control bead may be between about 10⁻³ to about 10³. The ratio of the first control bead to the second control bead may be between about 10⁻² to about 10². The ratio of the first control bead to the second control bead may be between about 10⁻¹ to about 10³. The ratio of the first control bead to the second control bead may be between about 1 to about 5. The ratio of the first control bead to the second control bead may be at least about 10⁻¹. The ratio of the first control bead to the second control bead may be at least about 10⁻¹. The ratio of the first control bead to the second control bead may be at least about 0.5. The ratio of the first control bead to the second control bead may be at least about 1. The ratio of the first control bead to the second control bead may be at least about 5. The ratio of the first control bead to the second control bead may be at least about 10. The ratio of the first control bead to the second control bead may be at least about 10².

The herein described compositions, methods and kits may comprise one or more sets of control beads. A set of control beads may comprise one or more subset of control beads, e.g., a first subset of control beads and a second subset of control beads. A first set (or subset) of control beads and a second set (or subset) of control beads may be present in such a composition, methods, and/or kit in one or more ratios. Such a ratio may be at least about 1:0.001, 1:0.01, 1:0.1, 1:0.2, 1:0.3, 1:0.4, 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:1000, or more. Such a ratio may be at most about 1:1000, 1;100, 1:90, 1:80, 1:70, 1:60, 1:50, 1:40, 1:30, 1:20, 1:10, 1:9, 1:8, 1:7, 1:6, 1:5, 1:4, 1:3, 1:2, 1:1.9, 1:1.8, 1:1.7, 1:1.6, 1:1.5, 1:1.4, 1:1.3, 1:1.2, 1:1.1, 1:1, 1:0.9, 1:0.8, 1:0.7, 1:0.6, 1:0.5, 1:0.4, 1:0.3, 1:0.2, 1:0.1, 1:0.01, 1:0.001, or less.

The compositions, methods and kits of the present disclosure may comprise control beads. As described herein, such control beads may comprise two or more different control beads, wherein the two or more different control beads may be used or are present in various ratios as further described herein. The two or more different control beads may each comprise (or be functionalized with) two or more different nucleic acid molecules, wherein such two or more different nucleic acid molecules of a synthetic cell may be used or are present in various ratios within the synthetic cell or on the surface of the control bead. Thus, the compositions, methods and kits of the present disclosure may use such control beads in a variety of ratios and amounts. For example, a composition, method, and/or kit may comprise two control beads (or two sets or subsets of control beads), wherein each of the two control beads comprises or is functionalized with about 96 different nucleic acid molecules (e.g., RNA molecules). Said two control beads may be used in certain, defined ratios. In some cases, such ratio is about 1:2. Similarly, each of the about 96 different nucleic acid molecules that the control bead may be functionalized with may be present within (and/or on the surface of) said control bead or on the surface of said control bead in certain, defined ratios. Said two control beads may be used in a method or kit as described herein.

A control bead disclosed herein may mimic single cell behavior in single cell processing experiments. As described herein, said control bead may be a bead comprising one or more known sequences. The bead may be a gel bead. The control bead may be introduced into a cell sample and have approximately the same or substantially the same size as other cells in the cell sample. The control bead may be carried through the entire workflow of a single cell processing experiment with the cell sample. After sequencing, the one or more known sequences in the control bead may be identified and analyzed to determine various characteristics of the single cell processing experiment, such as the effectiveness or the efficiency of the library preparation process or the sequencing process. Thus, the control beads (e.g., synthetic cells) described herein may be used in library preparation processes and/or in sequencing experiments and may be used as control standards that allow such processes to be performed with increased accuracy and efficiency compared to conventional methodologies. For example, control beads may be particularly useful in the analysis and processing of biological samples that contain cells of more than one species, such as human and mouse cells (e.g., barnyard experiments). For example, a control bead of the present disclosure may be used to account for PCR chimeras that may be produced during such barnyard experiments.

The compositions, methods and kits of the present disclosure may comprise barcode-containing beads and/or control beads, or any combination thereof (e.g., synthetic cells) in partitions (e.g., droplets or wells) for analyzing biological samples (e.g., single-cell analysis). The control beads may act as a quality control element and/or internal standard to improve accuracy and efficiency of single-cell analyses, and address the shortcomings of current methodologies as further described herein. For example, a control bead of the present disclosure may be used to determine a doublet rate.

### Reagents

In accordance with certain aspects, analyte carriers may be partitioned along with lysis reagents in order to release the contents of the analyte carriers within the partition. In such cases, the lysis agents can be contacted with the analyte carrier suspension concurrently with, or immediately prior to, the introduction of the analyte carriers into the partitioning junction/droplet generation zone (e.g., junction 210), such as through an additional channel or channels upstream of the channel junction. In accordance with other aspects, additionally or alternatively, analyte carriers may be partitioned along with other reagents, as will be described further below.

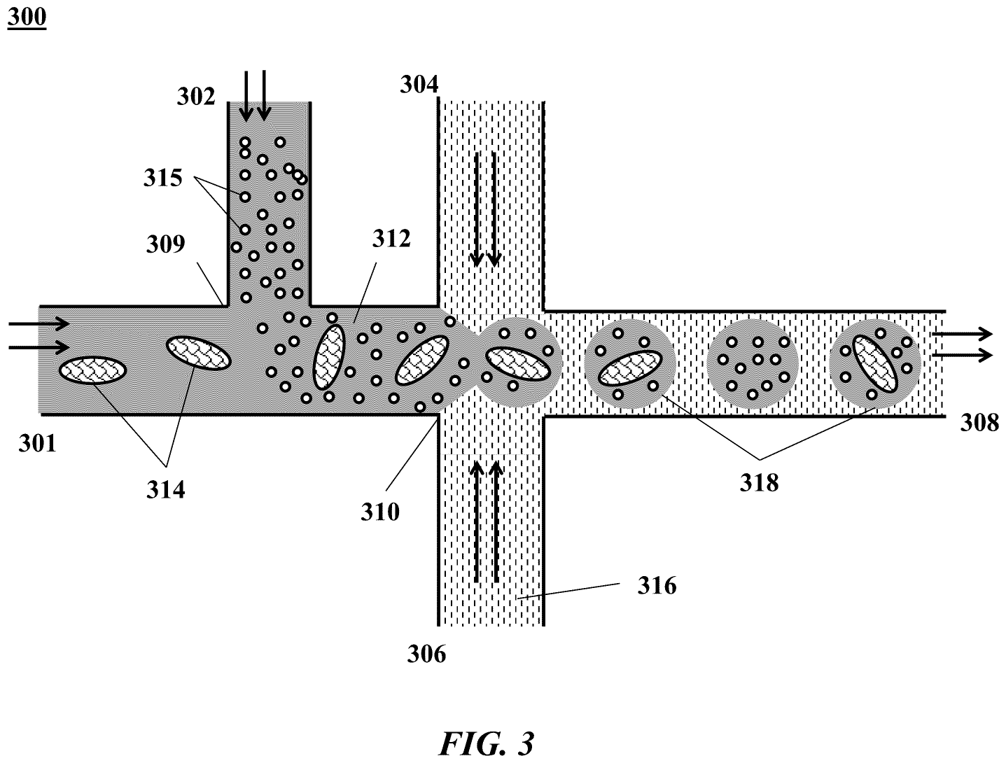

FIG. 3 shows an example of a microfluidic channel structure 300 for co-partitioning analyte carriers and reagents. The channel structure 300 can include channel segments 301, 302, 304, 306 and 308. Channel segments 301 and 302 communicate at a first channel junction 309. Channel segments 302, 304, 306, and 308 communicate at a second channel junction 310.

In an example operation, the channel segment 301 may transport an aqueous fluid 312 that includes a plurality of analyte carriers 314 along the channel segment 301 into the second junction 310. As an alternative or in addition to, channel segment 301 may transport beads (e.g., gel beads). The beads may comprise barcode molecules.

For example, the channel segment 301 may be connected to a reservoir comprising an aqueous suspension of analyte carriers 314. Upstream of, and immediately prior to reaching, the second junction 310, the channel segment 301 may meet the channel segment 302 at the first junction 309. The channel segment 302 may transport a plurality of reagents 315 (e.g., lysis agents) suspended in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the analyte carriers 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 can be partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be harvested.

The second fluid 316 can comprise an oil, such as a fluorinated oil, that includes a fluorosurfactant for stabilizing the resulting droplets, for example, inhibiting subsequent coalescence of the resulting droplets 318.

A discrete droplet generated may include an individual analyte carrier 314 and/or one or more reagents 315. In some instances, a discrete droplet generated may include a barcode carrying bead (not shown), such as via other microfluidics structures described elsewhere herein. In some instances, a discrete droplet may be unoccupied (e.g., no reagents, no analyte carriers).

Beneficially, when lysis reagents and analyte carriers are co-partitioned, the lysis reagents can facilitate the release of the contents of the analyte carriers within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or analyte carriers that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the analyte carriers to cause the release of the analyte carriers's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of analyte carriers that may be in addition to or in place of droplet partitioning, where any pore size of the encapsulate is sufficiently small to retain nucleic acid fragments of a given size, following cellular disruption.

Alternatively or in addition to the lysis agents co-partitioned with the analyte carriers described above, other reagents can also be co-partitioned with the analyte carriers, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated analyte carriers, the analyte carriers may be exposed to an appropriate stimulus to release the analyte carriers or their contents from a co-partitioned microcapsule. For example, in some cases, a chemical stimulus may be co-partitioned along with an encapsulated analyte carrier to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated analyte carrier to be released into a partition at a different time from the release of nucleic acid molecules into the same partition.

Additional reagents may also be co-partitioned with the analyte carriers, such as endonucleases to fragment an analyte carrier's DNA, DNA polymerase enzymes and dNTPs used to amplify the analyte carrier's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region.

The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxy Inosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of analyte carriers, such as RNA, DNA, or proteins) contained therein may be further processed within the partitions. In accordance with the methods and systems described herein, the macromolecular component contents of individual analyte carriers can be provided with unique identifiers such that, upon characterization of those macromolecular components they may be attributed as having been derived from the same analyte carrier or particles. The ability to attribute characteristics to individual analyte carriers or groups of analyte carriers is provided by the assignment of unique identifiers specifically to an individual analyte carrier or groups of analyte carriers. Unique identifiers, e.g., in the form of nucleic acid barcodes can be assigned or associated with individual analyte carriers or populations of analyte carriers, in order to tag or label the analyte carrier's macromolecular components (and as a result, its characteristics) with the unique identifiers. These unique identifiers can then be used to attribute the analyte carrier's components and characteristics to an individual analyte carrier or group of analyte carriers.

In some aspects, this is performed by co-partitioning the individual analyte carrier or groups of analyte carriers with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual analyte carrier, or to other components of the analyte carrier, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned analyte carriers. These sequences include, e.g., targeted or random/universal amplification primer sequences for amplifying the genomic DNA from the individual analyte carriers within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of analyte carriers, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

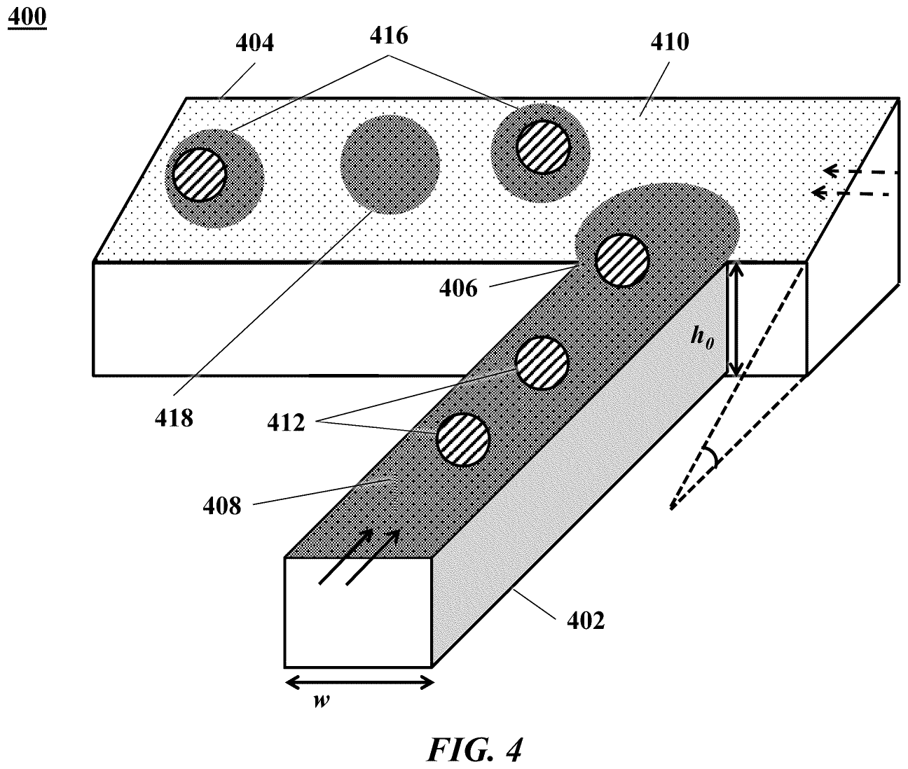

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the junction 406.

A discrete droplet generated may include a bead (e.g., as in occupied droplets 416). Alternatively, a discrete droplet generated may include more than one bead. Alternatively, a discrete droplet generated may not include any beads (e.g., as in unoccupied droplet 418). In some instances, a discrete droplet generated may contain one or more analyte carriers, as described elsewhere herein. In some instances, a discrete droplet generated may comprise one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can comprise analyte carriers (e.g., described with reference to FIGS. 1 and 2). In some instances, the aqueous fluid 408 can have a substantially uniform concentration or frequency of analyte carriers. As with the beads, the analyte carriers can be introduced into the channel segment 402 from a separate channel. The frequency or concentration of the analyte carriers in the aqueous fluid 408 in the channel segment 402 may be controlled by controlling the frequency in which the analyte carriers are introduced into the channel segment 402 and/or the relative flow rates of the fluids in the channel segment 402 and the separate channel. In some instances, the analyte carriers can be introduced into the channel segment 402 from a plurality of different channels, and the frequency controlled accordingly. In some instances, a first separate channel can introduce beads and a second separate channel can introduce analyte carriers into the channel segment 402. The first separate channel introducing the beads may be upstream or downstream of the second separate channel introducing the analyte carriers.

The second fluid 410 can comprise an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 may be substantially stationary in the reservoir 404. In some instances, the second fluid 410 may be subjected to flow within the reservoir 404, but not in or out of the reservoir 404, such as via application of pressure to the reservoir 404 and/or as affected by the incoming flow of the aqueous fluid 408 at the junction 406. Alternatively, the second fluid 410 may be subjected and/or directed to flow in or out of the reservoir 404. For example, the reservoir 404 can be a channel directing the second fluid 410 from upstream to downstream, transporting the generated droplets.

The channel structure 400 at or near the junction 406 may have certain geometric features that at least partly determine the sizes of the droplets formed by the channel structure 400. The channel segment 402 can have a height, h₀ and width, w, at or near the junction 406. By way of example, the channel segment 402 can comprise a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the junction 406 can be inclined at an expansion angle, α. The expansion angle, α, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 um, h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 um. In some instances, the flow rate of the aqueous fluid 408 entering the junction 406 can be between about 0.04 microliters (μL)/minute (min) and about 40 μL/min. In some instances, the flow rate of the aqueous fluid 408 entering the junction 406 can be between about 0.01 microliters (μL)/minute (min) and about 100 μL/min. Alternatively, the flow rate of the aqueous fluid 408 entering the junction 406 can be less than about 0.01 μL/min. Alternatively, the flow rate of the aqueous fluid 408 entering the junction 406 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 408 entering the junction 406.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 406) between aqueous fluid 408 channel segments (e.g., channel segment 402) and the reservoir 404. Alternatively or in addition, the throughput of droplet generation can be increased by increasing the flow rate of the aqueous fluid 408 in the channel segment 402.

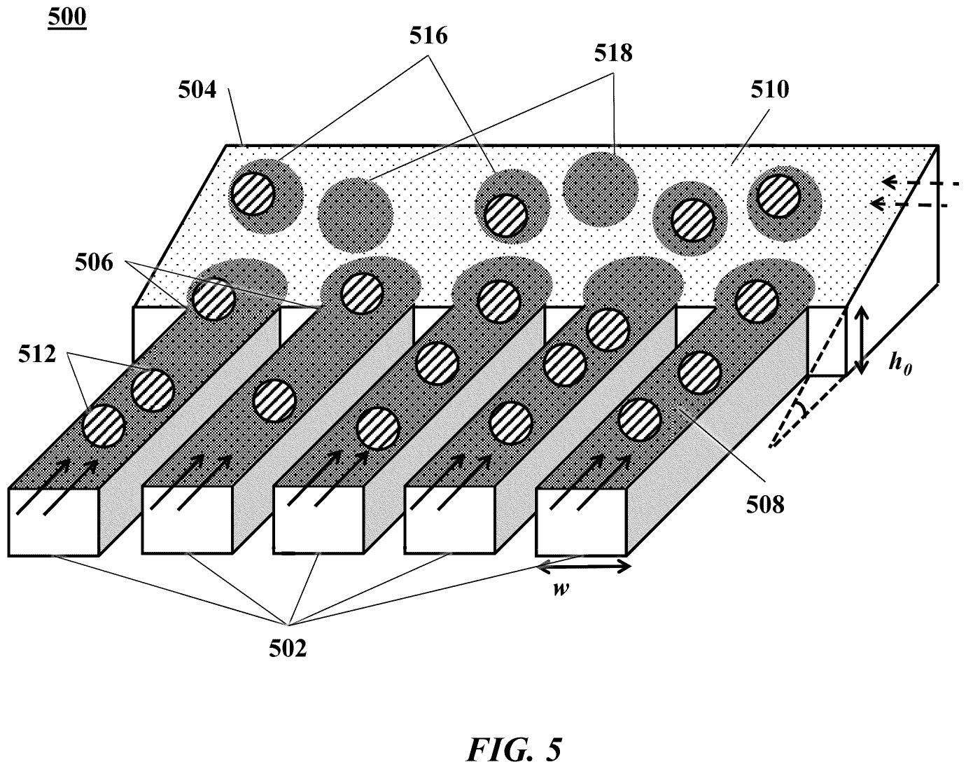

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

Each channel segment of the plurality of channel segments 502 may comprise an aqueous fluid 508 that includes suspended beads 512. The reservoir 504 may comprise a second fluid 510 that is immiscible with the aqueous fluid 508. In some instances, the second fluid 510 may not be subjected to and/or directed to any flow in or out of the reservoir 504. For example, the second fluid 510 may be substantially stationary in the reservoir 504. In some instances, the second fluid 510 may be subjected to flow within the reservoir 504, but not in or out of the reservoir 504, such as via application of pressure to the reservoir 504 and/or as affected by the incoming flow of the aqueous fluid 508 at the junctions. Alternatively, the second fluid 510 may be subjected and/or directed to flow in or out of the reservoir 504. For example, the reservoir 504 can be a channel directing the second fluid 510 from upstream to downstream, transporting the generated droplets.

In operation, the aqueous fluid 508 that includes suspended beads 512 may be transported along the plurality of channel segments 502 into the plurality of junctions 506 to meet the second fluid 510 in the reservoir 504 to create droplets 516, 518. A droplet may form from each channel segment at each corresponding junction with the reservoir 504. At the junction where the aqueous fluid 508 and the second fluid 510 meet, droplets can form based on factors such as the hydrodynamic forces at the junction, flow rates of the two fluids 508, 510, fluid properties, and certain geometric parameters (e.g., w, h₀, α, etc.) of the channel structure 500, as described elsewhere herein. A plurality of droplets can be collected in the reservoir 504 by continuously injecting the aqueous fluid 508 from the plurality of channel segments 502 through the plurality of junctions 506. Throughput may significantly increase with the parallel channel configuration of channel structure 500. For example, a channel structure having five inlet channel segments comprising the aqueous fluid 508 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and α, may or may not be uniform for each of the channel segments in the plurality of channel segments 502. For example, each channel segment may have the same or different widths at or near its respective channel junction with the reservoir 504. For example, each channel segment may have the same or different height at or near its respective channel junction with the reservoir 504. In another example, the reservoir 504 may have the same or different expansion angle at the different channel junctions with the plurality of channel segments 502. When the geometric parameters are uniform, beneficially, droplet size may also be controlled to be uniform even with the increased throughput. In some instances, when it is desirable to have a different distribution of droplet sizes, the geometric parameters for the plurality of channel segments 502 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

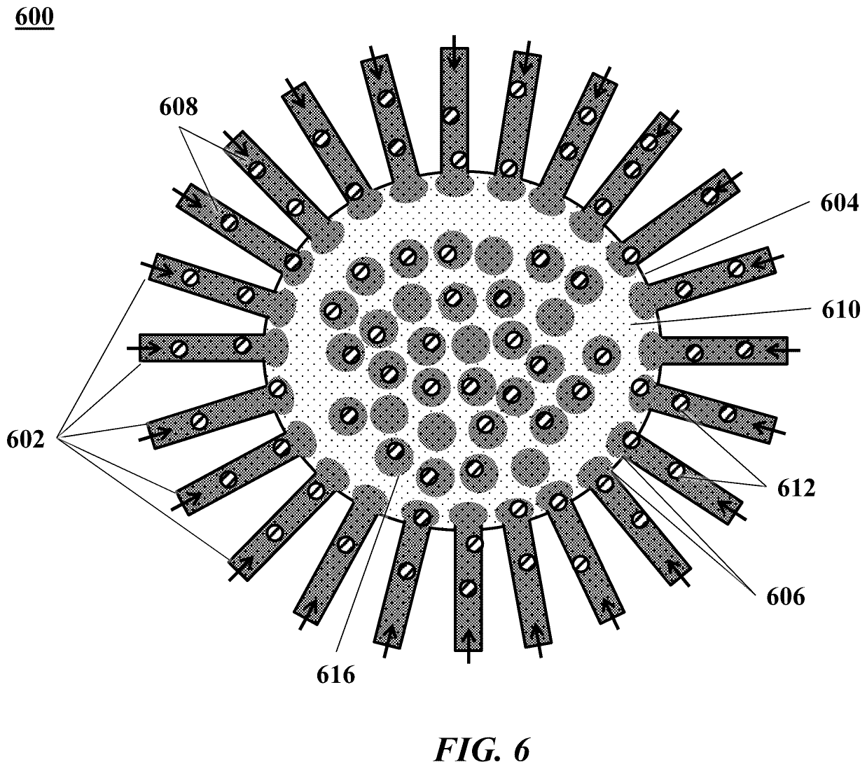

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 2 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof.

Each channel segment of the plurality of channel segments 602 may comprise an aqueous fluid 608 that includes suspended beads 612. The reservoir 604 may comprise a second fluid 610 that is immiscible with the aqueous fluid 608. In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junctions. Alternatively, the second fluid 610 may be subjected and/or directed to flow in or out of the reservoir 604. For example, the reservoir 604 can be a channel directing the second fluid 610 from upstream to downstream, transporting the generated droplets. In operation, the aqueous fluid 608 that includes suspended beads 612 may be

transported along the plurality of channel segments 602 into the plurality of junctions 606 to meet the second fluid 610 in the reservoir 604 to create a plurality of droplets 616. A droplet may form from each channel segment at each corresponding junction with the reservoir 604. At the junction where the aqueous fluid 608 and the second fluid 610 meet, droplets can form based on factors such as the hydrodynamic forces at the junction, flow rates of the two fluids 608, 610, fluid properties, and certain geometric parameters (e.g., widths and heights of the channel segments 602, expansion angle of the reservoir 604, etc.) of the channel structure 600, as described elsewhere herein. A plurality of droplets can be collected in the reservoir 604 by continuously injecting the aqueous fluid 608 from the plurality of channel segments 602 through the plurality of junctions 606. Throughput may significantly increase with the substantially parallel channel configuration of the channel structure 600. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 604 may have an expansion angle, α (not shown in FIG. 6) at or near each channel junction. Each channel segment of the plurality of channel segments 602 may have a width, w, and a height, h₀, at or near the channel junction. The geometric parameters, w, h₀, and α, may or may not be uniform for each of the channel segments in the plurality of channel segments 602. For example, each channel segment may have the same or different widths at or near its respective channel junction with the reservoir 604. For example, each channel segment may have the same or different height at or near its respective channel junction with the reservoir 604.

The reservoir 604 may have the same or different expansion angle at the different channel junctions with the plurality of channel segments 602. For example, a circular reservoir (as shown in FIG. 6) may have a conical, dome-like, or hemispherical ceiling (e.g., top wall) to provide the same or substantially same expansion angle for each channel segments 602 at or near the plurality of channel junctions 606. When the geometric parameters are uniform, beneficially, resulting droplet size may be controlled to be uniform even with the increased throughput. In some instances, when it is desirable to have a different distribution of droplet sizes, the geometric parameters for the plurality of channel segments 602 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or analyte carrier injected into the droplets may or may not have uniform size.

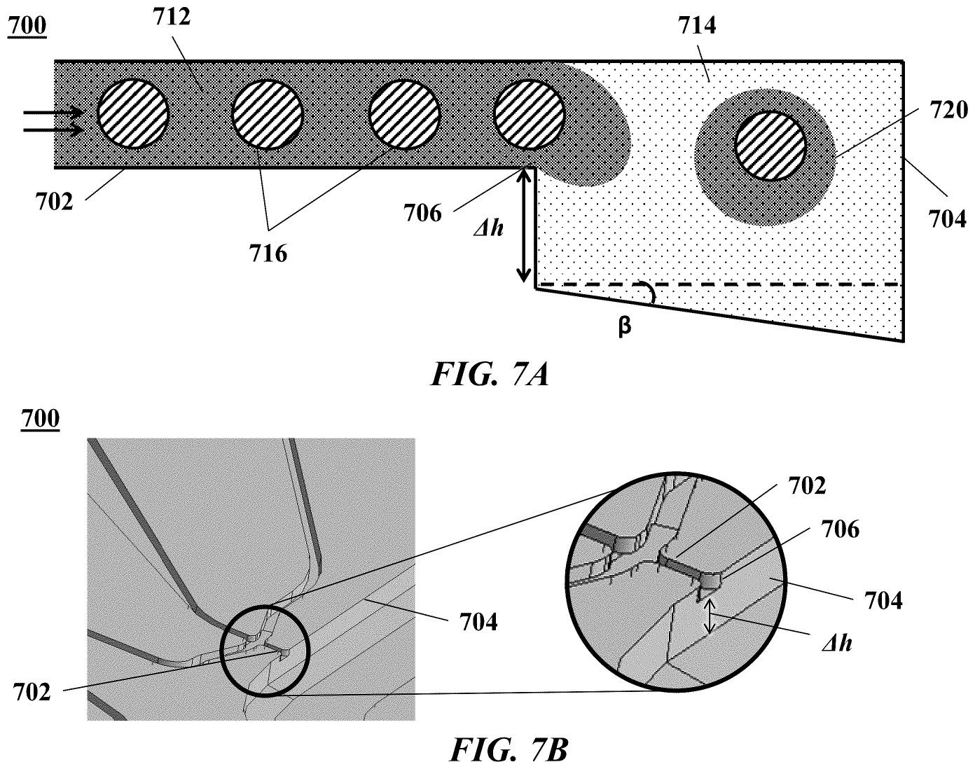

FIG. 7A shows a cross-section view of another example of a microfluidic channel structure with a geometric feature for controlled partitioning. A channel structure 700 can include a channel segment 702 communicating at a channel junction 706 (or intersection) with a reservoir 704. In some instances, the channel structure 700 and one or more of its components can correspond to the channel structure 100 and one or more of its components. FIG. 7B shows a perspective view of the channel structure 700 of FIG. 7A.

An aqueous fluid 712 comprising a plurality of particles 716 may be transported along the channel segment 702 into the junction 706 to meet a second fluid 714 (e.g., oil, etc.) that is immiscible with the aqueous fluid 712 in the reservoir 704 to create droplets 720 of the aqueous fluid 712 flowing into the reservoir 704. At the junction 706 where the aqueous fluid 712 and the second fluid 714 meet, droplets can form based on factors such as the hydrodynamic forces at the junction 706, relative flow rates of the two fluids 712, 714, fluid properties, and certain geometric parameters (e.g., Δh, etc.) of the channel structure 700. A plurality of droplets can be collected in the reservoir 704 by continuously injecting the aqueous fluid 712 from the channel segment 702 at the junction 706.

A discrete droplet generated may comprise one or more particles of the plurality of particles 716. As described elsewhere herein, a particle may be any particle, such as a bead, cell bead, gel bead, analyte carrier, macromolecular constituents of analyte carrier, or other particles. Alternatively, a discrete droplet generated may not include any particles.

In some instances, the aqueous fluid 712 can have a substantially uniform concentration or frequency of particles 716. As described elsewhere herein (e.g., with reference to FIG. 4), the particles 716 (e.g., beads) can be introduced into the channel segment 702 from a separate channel (not shown in FIG. 7). The frequency of particles 716 in the channel segment 702 may be controlled by controlling the frequency in which the particles 716 are introduced into the channel segment 702 and/or the relative flow rates of the fluids in the channel segment 702 and the separate channel. In some instances, the particles 716 can be introduced into the channel segment 702 from a plurality of different channels, and the frequency controlled accordingly. In some instances, different particles may be introduced via separate channels. For example, a first separate channel can introduce beads and a second separate channel can introduce analyte carriers into the channel segment 702. The first separate channel introducing the beads may be upstream or downstream of the second separate channel introducing the analyte carriers.

In some instances, the second fluid 714 may not be subjected to and/or directed to any flow in or out of the reservoir 704. For example, the second fluid 714 may be substantially stationary in the reservoir 704. In some instances, the second fluid 714 may be subjected to flow within the reservoir 704, but not in or out of the reservoir 704, such as via application of pressure to the reservoir 704 and/or as affected by the incoming flow of the aqueous fluid 712 at the junction 706. Alternatively, the second fluid 714 may be subjected and/or directed to flow in or out of the reservoir 704. For example, the reservoir 704 can be a channel directing the second fluid 714 from upstream to downstream, transporting the generated droplets.

The channel structure 700 at or near the junction 706 may have certain geometric features that at least partly determine the sizes and/or shapes of the droplets formed by the channel structure 700. The channel segment 702 can have a first cross-section height, h₁, and the reservoir 704 can have a second cross-section height, h₂. The first cross-section height, h₁, and the second cross-section height, h₂ may be different, such that at the junction 706, there is a height difference of Δh. The second cross-section height, h₂, may be greater than the first cross-section height, h₁. In some instances, the reservoir may thereafter gradually increase in cross-section height, for example, the more distant it is from the junction 706. In some instances, the cross-section height of the reservoir may increase in accordance with expansion angle, β, at or near the junction 706. The height difference, Δh, and/or expansion angle, β, can allow the tongue (portion of the aqueous fluid 712 leaving channel segment 702 at junction 706 and entering the reservoir 704 before droplet formation) to increase in depth and facilitate decrease in curvature of the intermediately formed droplet. For example, droplet size may decrease with increasing height difference and/or increasing expansion angle.

The height difference, Δh, can be at least about 1 μm. Alternatively, the height difference can be at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 μm or more. Alternatively, the height difference can be at most about 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1 μm or less. In some instances, the expansion angle, β, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less.

In some instances, the flow rate of the aqueous fluid 712 entering the junction 706 can be between about 0.04 microliters (μL)/minute (min) and about 40 μL/min. In some instances, the flow rate of the aqueous fluid 712 entering the junction 706 can be between about 0.01 microliters (μL)/minute (min) and about 100 μL/min. Alternatively, the flow rate of the aqueous fluid 712 entering the junction 706 can be less than about 0.01 μL/min. Alternatively, the flow rate of the aqueous fluid 712 entering the junction 706 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 712 entering the junction 706. The second fluid 714 may be stationary, or substantially stationary, in the reservoir 704. Alternatively, the second fluid 714 may be flowing, such as at the above flow rates described for the aqueous fluid 712.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

While FIGS. 7A and 7B illustrate the height difference, 4h, being abrupt at the junction 706 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, wherein an angle between any one differential segment of a height profile and an immediately adjacent differential segment of the height profile is greater than 90°. For example, at the junction 706, a bottom wall of the channel and a bottom wall of the reservoir can meet at an angle greater than 90°. Alternatively or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively or in addition, the height difference may variably increase and/or decrease linearly or non-linearly. While FIGS. 7A and 7B illustrate the expanding reservoir cross-section height as linear (e.g., constant expansion angle, β), the cross-section height may expand non-linearly. For example, the reservoir may be defined at least partially by a dome-like (e.g., hemispherical) shape having variable expansion angles. The cross-section height may expand in any shape.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different analyte carrier or organism types within a population of analyte carriers, including, for example, microbiome analysis and characterization, environmental testing, food safety testing, epidemiological analysis, e.g., in tracing contamination or the like.

### Computer Systems

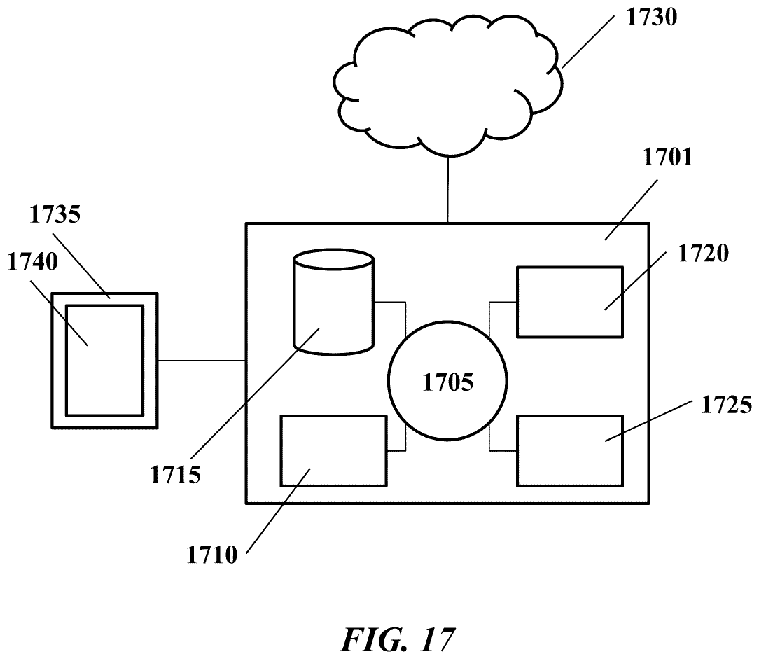

The present disclosure provides computer systems that are programmed to implement methods of the disclosure. FIG. 17 shows a computer system 1701 that is programmed or otherwise configured to (i) control a microfluidics system (e.g., fluid flow), (ii) sort occupied droplets from unoccupied droplets, (iii) polymerize droplets, (iv) perform sequencing applications, (v) generate and maintain a library of barcoded analytes, and (vi) analyze results, such as to determine doublet rate or UMI purity. The computer system 1701 can be an electronic device of a user or a computer system that is remotely located with respect to the electronic device. The electronic device can be a mobile electronic device.

The computer system 1701 includes a central processing unit (CPU, also “processor” and “computer processor” herein) 1705, which can be a single core or multi core processor, or a plurality of processors for parallel processing. The computer system 1701 also includes memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720 and peripheral devices 1725 are in communication with the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can include one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can include fetch, decode, execute, and writeback.

The CPU 1705 can be part of a circuit, such as an integrated circuit. One or more other components of the system 1701 can be included in the circuit. In some cases, the circuit is an application specific integrated circuit (ASIC).

The storage unit 1715 can store files, such as drivers, libraries and saved programs. The storage unit 1715 can store user data, e.g., user preferences and user programs. The computer system 1701 in some cases can include one or more additional data storage units that are external to the computer system 1701, such as located on a remote server that is in communication with the computer system 1701 through an intranet or the Internet.

The computer system 1701 can communicate with one or more remote computer systems through the network 1730. For instance, the computer system 1701 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or machine readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can include or be in communication with an electronic display 1735 that comprises a user interface (UI) 1740 for providing, for example, results of sequencing analysis, such as the doublet rate or UMI purity rate, or a control panel for one or more single cell application units or microfluidics units. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1705. The algorithm can, for example, generate sequencing results and process sequencing results to determine, for example doublet rate or UMi purity rate, or other parameters of a single cell application.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) form a single cell. For example, an analyte carrier (e.g., a cell or cell bead) is partitioned in a partition (e.g., droplet), and multiple analytes from the analyte carrier are processed for subsequent processing. The multiple analytes may be from the single cell. This may enable, for example, simultaneous proteomic, transcriptomic and genomic analysis of the cell.

### EXAMPLES

The following examples merely illustrate the disclosure, and are not intended to limit the disclosure in any way.

### Example 1

### Production of Ligated RNA Constructs

This example demonstrates the production and analysis of ligated RNA constructs that may be used to functionalize beads (e.g., gel beads that can be used as control beads).

RNA transcripts were first created from gblocks synthesized by Integrated DNA Technologies (IDT), the sequences were designed with a T7 primer on the 5′ end (e.g., TAATACGACTCACTATAGGG) to be used later for RNA synthesis via in vitro transcription. The sequences for the gblocks used in this experiment can be seen below but any sequence may be used. The following two, exemplary RNA transcripts were used:

(SEQ ID NO: 1, SynthCell #1)
TAATACGACTCACTATAGGGAGCGTCTGTTATGATTCGGTTGTACCCGG
AACAACTCCGTGCTCAGCTCAATGAAGGGCTGCGTGCAGCATATCTTTT
ACTTGGTAACGATCCTCTGTTATTGCAGGAAAGCCAGGACGCTGTTCGT
CAGGTAGCTGCGGCACAAGGATTCGAAGAACACCACACTTTTTCCATTG
ATCCCAACACTGACTGGAATGCGATCTTTTCGTTATGCCAGGCTATGAG
TCTGTTTGCCAGTCGACAAACGCTATTGCTGTTGTTACCAGAAAACGGA
CCGAATGCGGCGATCAATGAGCAACTTCTCACACTCACCGGACTTCTGC
ATGACGACCTGCTGTTGATCGTCCGCGGTAATAAATTAAGCAAAGCGCA
AGAAAATGCCGCCTGGTTTACTGCGCTTGCGAATCGCAGCGTGCAGGTG
ACCTGTCAGACACCGGAGCAGGCTCAGCTTCCCCGCTGGGTTGCTGCGT
AG,
and
(SEQ ID NO: 2, SynthCell #2)
TAATACGACTCACTATAGGGAGCGTCTCTTATCATTCGGTTGTACCCGG
AACAACTCCGATCGCAGCTCAATGAAGGGCTGCGCGATGCGTATCTTTT
ACTTGGTAACGATCCTCTGTTATTGCAG.

FIG. 11 shows how RNA was subsequently created from these transcripts in step 1101 using New England Biolabs HiScribe T7 ARCA mRNA Kit with tailing, however, any similar kit or kits may be used to complete in vitro transcription, capping and A-tailing. This process created an RNA sequence starting at the last guanine of the T7 primer and ending with 100-150 adenines on the 3′ end of the RNA. The RNA was cleaned and concentrated using the Zymo Clean and Concentrator kit but any kit may be used to clean the RNA. The concentration of synthesized RNA was then quantified using a Nanodrop analyzer.

Ligation of the RNA molecule to an azide group was completed using T4 DNA Ligase, a single stranded splint sequence containing 10 thymines and a 10 bp recognition sequence, and an oligo containing the complement 10 bp recognition sequence on the 5′ end and an azide group on the 3′ end as shown in step 1102 of FIG. 11. The DNA ligase may be from any manufacturer but the one used in the experiment came from Enzymatics and the stock was 500 mg/mL. Additionally, the oligos may be synthesized by any manufacturer but these were made by IDT.

The ligation reaction contained 10 uM synthesized RNA, 10 uM splint, 40 uM azide oligo, and 100 U/uL or 50 ug/uL ligase, Ligation Buffer was then added up to 250 uL. The reaction was incubated at 16 degrees For 1 hour before 3 uL of EDTA was added to stop the reaction. The reaction was then cleaned to remove small un-ligated oligos using the Zymo Clean and Concentrator Kit. The concentration of ligated RNA was then quantified using a Nanodrop analyzer.

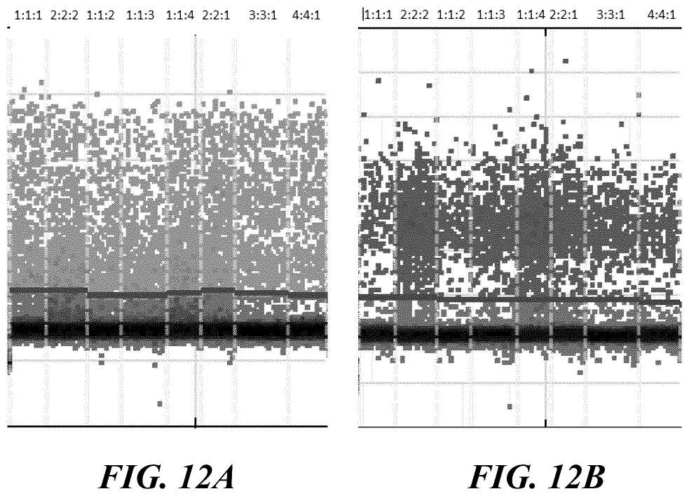

This ligation was validated through digital droplet PCR (ddPCR). Primer and probes were designed to amplify the full length ligated product and cause fluorescence. FIG. 12A (resulted from the first RNA transcript used) and FIG. 12B (resulted from the first RNA transcript used) show the results of a ddPCR testing different ratios of components in the ligation reaction with the first number representing the synthesized RNA, the second representing the splint and the third representing the azide oligo. The number of dots above the pink line denotes the number of full length ligated RNAs in the sample. Of the reaction ratios tested, the ratio of 1:1:4 appeared to have the highest number of positive events. The ligated RNA product was stored at −80 until used for clicking onto the gel beads (e.g., control beads).

This example demonstrates that nucleic acid molecules (e.g., RNA molecules) may be efficiently functionalized with various moieties, such as those enabling functionalization of beads such as control beads.

Example 2

### Production of RNA-Functionalized Gel Beads

This example demonstrates the production and analysis of RNA-functionalized gel beads (e.g., those that can be used as control beads) using azide-modified RNA samples, e.g., those that were produced as described above in EXAMPLE 1.

First, 30 μm RNA-functionalized gel beads were produced using methods known in the art (see, e.g., U.S. Pat. Nos. 9,695,468, 9,951,386, and 9,689,024 and U.S. Patent Application Publication Nos. 2015/0376609, 2017/0321252, 2018/0016634, 2018/0216162, and 2018/0371540, which are incorporated herein by reference in their entirety) with the exception that the primer on the gel bead was replaced with a primer containing an alkyne group which could be used later in the click chemistry reaction. Additionally, Bis-acrylamide was used in place of Bis-Cys to keep the beads from dissolving in DTT. The aqueous solution was formulated so that the final gel beads had an alkyne concentration of 20 uM.

The functionalized gel beads were also run through the flow cam to verify the size, these functionalized gel beads did not swell as much as unmodified genome gel beads, the average sizes over three runs measuring 25,000-30,000 gel beads per run was 28.24 um. The smaller size is likely due to the replacement of bis-cys for bis-acrylamide.

Click chemistry was used to attach the azide-functionalized RNA constructs onto the beads and was completed using stock solutions of the ligand THPTA at 50 mM, 10 mM copper acetate, 100 mM sodium ascorbate, 0.16 mM ligated RNA, and the 20 uM gel beads (see e.g., step 1301 shown in FIG. 13). The final reaction was 300 uL 1.25 mM THPTA, 0.25 mM CuAcO₄, 5.0 mM sodium ascorbate and 0.04 mM ligated RNA, and 20 uM gel beads. This reaction was incubated at room temperature while rolling at 30 rpm for 2 hours, then 4 uL of EDTA were added and the gel beads were washed in buffer.

Validation of the click chemistry reaction and thus validation of attachment of the RNA molecules to the beads was completed through probe hybridization of the Poly-A tail using a Poly-T oligo with an Alexa647 probe synthesized by IDT. 50 uL of the washed and packed gel beads were mixed with 50 uL of 12 uM polyT probe. The reaction was placed on a thermomixer at 95° C. and covered with a lid to protect the probe from light. The thermomixer was then set to 25° C. and 1000 rpm and the reaction was slowly cooled while shaking. After cooling the mixture was cleaned again with buffer to remove unclicked RNA and probe. 5 uL of the mixture were added to 1495 uL buffer in a Guava analysis tube which were mixed by vortexing then placed in the Guava machine. Three technical replicates were tested. If the ligation and click chemistry were successful there should be a polyA sequence attached to the gel bead which would then hybridize to the PolyT probe producing increased fluorescence visible on the Guava.

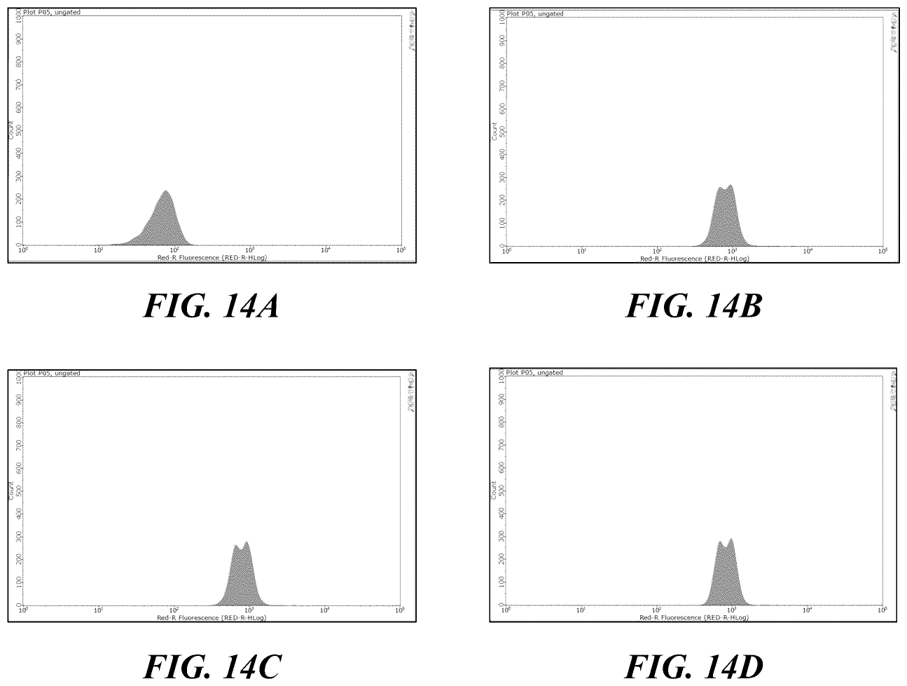

FIGS. 14A-D show the results of this test on the Guava, the location of the peak on the X-axis denotes level of red fluorescence. The negative control (FIG. 14A) went through the ligation and clicking processes but did not include an azide so no clicking would be possible but there was a PolyA sequence in the mix. The observed right shift in the fluorescence signal (FIG. 14A-C) which indicated an increase in fluorescence confirmed that the gel beads did contain the synthesized RNA and thus indicated attachment of nucleic acid molecules to the beads.

This example demonstrates that the azide-functionalized RNA molecules produced in EXAMPLE 1 can be efficiently attached (e.g., chemically linked via click chemistry and/or covalent or non-covalent bond formation) to gel beads. Such functionalized gel beads may be used as control beads, for example, as control elements in single-cell analyses processes.

### Example 3

### Use of RNA-Functionalized Gel Beads in Single-Cell Sequencing

This example demonstrates the use of synthetic cells as produced above in EXAMPLE 2 comprising RNA molecule(s) (e.g., attached to their surface) in a single-cell sequencing experiment to determine whether the transcripts can be detected after sequencing.

The synthetic cells used in the experiment were generated using the transcript sequences described in EXAMPLE 1. The ratio of the transcripts with SEQ ID NO: 1 and SEQ ID NO: 2 on a bead was about 1:2.

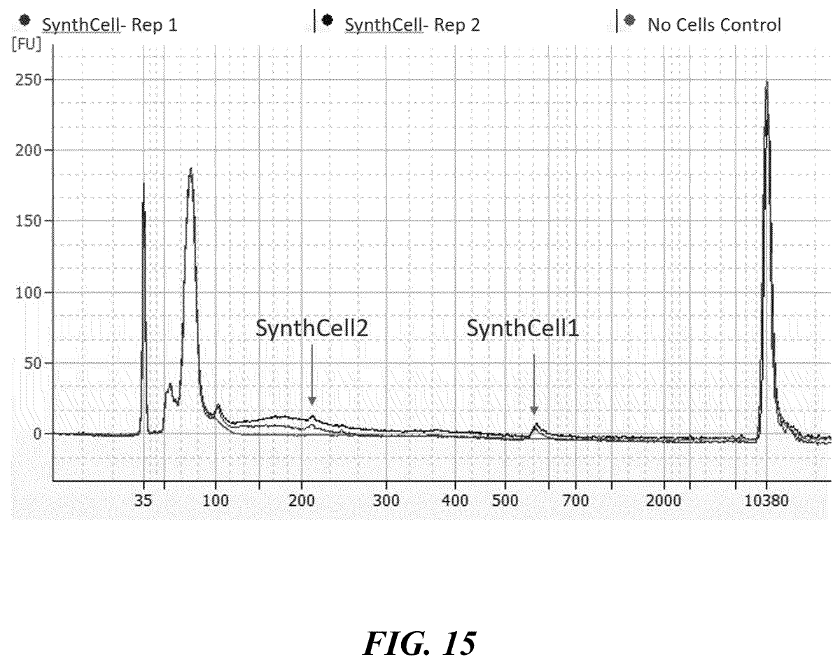

For this study, approximately 1000 synthetic cells were used. cDNA was produced and the size of the peaks matched the expected transcript sizes, with an additional peak at 75 bp which is likely primer dimer. The bioanalyzer traces are shown in FIG. 15.

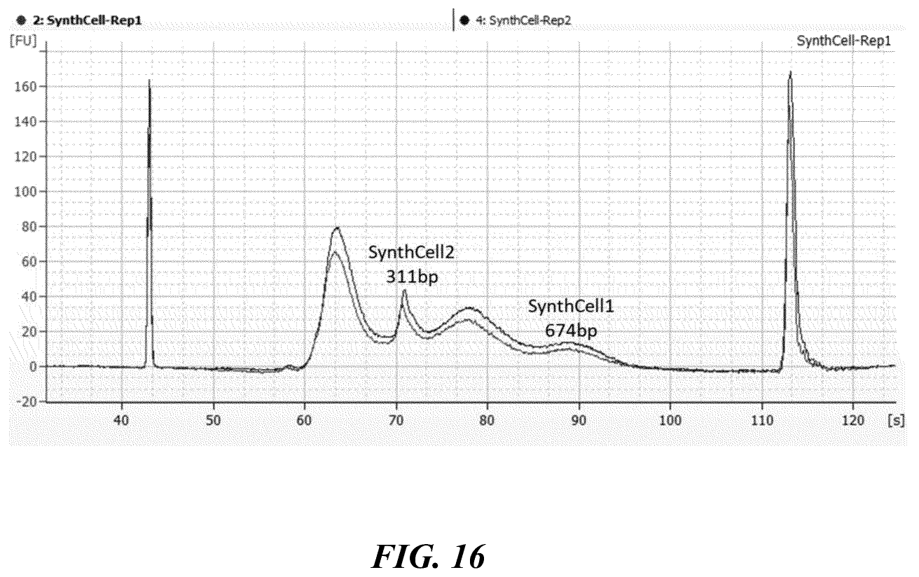

Libraries were then made from the cDNA and the corresponding bioanalyzer trace in FIG. 16 shows the results. The cDNA sequences were randomly fragmented, possibly explaining the peak width observed in this study. The first two peaks in the test samples and the first negative control are likely from the primer dimer seen in the cDNA, while the second two peaks, corresponding to 311 bp and 674 bp, respectively, were seen only in the test samples, and thus correspond with a high likelihood to the expected transcripts.

Subsequently, both test sample's libraries were submitted for sequencing on the MiSeq analyzer. Both samples produced high numbers of R2 sequence but 3% of the reads mapped to a part of one or both transcripts. The transcript sequences (see EXAMPLE 1) may have been too similar in their sequence to determine the ratio between the two sequences, however, the expected transcripts were clearly observed in the sequencing analysis.

Thus, the data presented herein demonstrate that the synthetic cells described herein can be used as a library preparation tool and/or as sequencing standards in single-cell analyses biological samples. The data further show that a nucleic acid sequence that is attached to a gel bead or control bead via click chemistry can be recovered during sequencing as if the nucleic acid sequence belonged to a cell (e.g., a cell of a biological sample to be analyzed in a single-cell analysis).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for nucleic acid processing, comprising:
(a) providing:
(i) a biological particle comprising (1) a vector encoding a guide ribonucleic acid (gRNA) sequence or a gRNA molecule comprising said gRNA sequence and (2) a deoxyribonucleic acid (DNA) molecule;
(ii) a first nucleic acid barcode molecule and a second nucleic acid barcode molecule, each comprising (1) a barcode sequence; and (2) an adapter sequence;
(iii) a first primer molecule comprising a first sequence complementary to said adapter sequence and a second sequence complementary to a sequence of said vector or said gRNA sequence; and
(iv) a second primer molecule comprising a sequence complementary to a sequence of said vector or said gRNA sequence;
(b) using (i) said first primer molecule, said second primer molecule, and said vector or said gRNA molecule to generate a first product comprising a sequence complementary to said gRNA sequence and said sequence complementary to said adapter sequence;
(c) using said first product or a derivative thereof and said first nucleic acid barcode molecule to generate a first barcoded nucleic acid molecule comprising (1) said barcode sequence or a reverse complement thereof and (2) said gRNA sequence or a reverse complement thereof; and
(d) using said DNA molecule or a derivative thereof and said second nucleic acid barcode molecule to generate a second barcoded nucleic acid molecule comprising (1) said barcode sequence or a reverse complement thereof and (2) a sequence of said DNA molecule.

2. The method of claim 1, wherein said biological particle is a cell, cell bead, or cell nucleus.

3. The method of claim 1, further comprising processing an open chromatin structure of said biological particle with a transposase to yield said DNA molecule comprising said adapter sequence or a portion thereof.

4. The method of claim 1, wherein said biological particle, said first nucleic acid barcode molecule, said second nucleic acid barcode molecule, said first primer molecule, and second primer molecule are provided in a partition.

5. The method of claim 3, wherein said transposase is included in a transposase-nucleic acid complex that comprises (i) a first transposase nucleic acid molecule comprising a first transposon end sequence and said adapter sequence or a reverse complement thereof and (ii) a second transposase nucleic acid molecule comprising a second transposon end sequence and a second adapter sequence.

6. The method of claim 5, wherein said first adapter sequence is a first sequencing primer or a portion thereof and (ii) said second adapter sequence is a second sequencing primer or a portion thereof.

7. The method of claim 6, wherein said second primer molecule further comprises said second adapter sequence or a reverse complement thereof.

8. The method of claim 3, wherein said first nucleic acid barcode molecule and said second nucleic acid barcode molecule comprise a common barcode sequence.

9. The method of claim 1, further comprising, prior to (a), delivering said vector to said biological particle.

10. The method of claim 1, wherein said vector is a plasmid vector or viral vector.

11. The method of claim 1, wherein said vector or said gRNA molecule comprises a barcode sequence that identifies said gRNA sequence.

12. A composition comprising a partition comprising:
(1) a biological particle comprising (i) a vector encoding a guide ribonucleic acid (gRNA) sequence or a gRNA molecule comprising said gRNA sequence, (ii) a deoxyribonucleic acid (DNA) molecule;
(2) a first nucleic acid barcode molecule and a second nucleic acid barcode molecule, each comprising (i) a barcode sequence and (ii) an adapter sequence;
(3) a first primer molecule comprising a first sequence complementary to said adapter sequence and a second sequence complementary to a sequence of said vector or said gRNA sequence; and
(4) a second primer molecule comprising a sequence complementary to a sequence of said vector or said gRNA sequence.

13. The composition of claim 12, wherein said biological particle is a cell, cell bead or cell nucleus.

14. The composition of claim 12, wherein said partition is a droplet or a well.

15. The composition of claim 14, wherein said well is a microwell.

16. The composition of claim 12, further comprising a transposase.

17. The composition of claim 12, wherein said vector is a plasmid vector or viral vector.

18. The composition of claim 12, wherein said vector or said gRNA molecule comprises a barcode sequence that identifies said gRNA sequence.

19. The composition of claim 12, wherein the first nucleic acid barcode molecule and said second nucleic acid barcode molecule are coupled to a bead.

20. The composition of claim 19, wherein said bead is a gel bead.


SEQUENCE LISTING (6 sequences)

SEQ ID NO: 1 - DNA, 492 bp, synthetic construct
  misc_feature 1..492: note = source = /note="Description of Artificial Sequence: Synthetic polynucleotide"
  source 1..492: mol_type = other DNA
taatacgactcactatagggagcgtctgttatgattcggttgtacccggaacaactccgtgctcagctcaatgaagggctgcgtgcagcatatcttttacttggtaacgatcctctgttattgcaggaaagccaggacgctgttcgtcaggtagctgcggcacaaggattcgaagaacaccacactttttccattgatcccaacactgactggaatgcgatcttttcgttatgccaggctatgagtctgtttgccagtcgacaaacgctattgctgttgttaccagaaaacggaccgaatgcggcgatcaatgagcaacttctcacactcaccggacttctgcatgacgacctgctgttgatcgtccgcggtaataaattaagcaaagcgcaagaaaatgccgcctggtttactgcgcttgcgaatcgcagcgtgcaggtgacctgtcagacaccggagcaggctcagcttccccgctgggttgctgcgtag

SEQ ID NO: 2 - DNA, 126 bp, synthetic construct
  misc_feature 1..126: note = source = /note="Description of Artificial Sequence: Synthetic polynucleotide"
  source 1..126: mol_type = other DNA
taatacgactcactatagggagcgtctcttatcattcggttgtacccggaacaactccgatcgcagctcaatgaagggctgcgcgatgcgtatcttttacttggtaacgatcctctgttattgcag

SEQ ID NO: 3 - DNA, 20 bp, synthetic construct
  misc_feature 1..20: note = source = /note="Description of Artificial Sequence: Synthetic primer"
  source 1..20: mol_type = other DNA
taatacgactcactataggg

SEQ ID NO: 4 - DNA, 15 bp, synthetic construct
  misc_feature 1..15: note = source = /note="Description of Artificial Sequence: Synthetic oligonucleotide"
  source 1..15: mol_type = other DNA
tagaaaaaaaaaaaa

SEQ ID NO: 5 - DNA, 23 bp, synthetic construct
  misc_feature 1..23: note = source = /note="Description of Artificial Sequence: Synthetic oligonucleotide"
  source 1..23: mol_type = other DNA
aaaaaaaaaaaaaaaaaaaaaaa

SEQ ID NO: 6 - DNA, 21 bp, synthetic construct
  misc_difference 1..10: note = a, t, c, g, unknown, or other
  misc_feature 1..21: note = source = /note="Description of Artificial Sequence: Synthetic oligonucleotide"
  source 1..21: mol_type = other DNA
nnnnnnnnnnttttttttttt